(19) United States
(12) Patent Application Publication
(10) Pub. No.: US 2020/0188920 A1
(43) Pub. Date: Jun. 18, 2020

(54) METHODS AND SYSTEMS FOR IMPROVED DROPLET STABILIZATION

(71) Applicant: 10X GENOMICS, INC., Pleasanton, CA (US)

(72) Inventors: Joshua Delaney, Pleasanton, CA (US); Andrew D. Price, Pleasanton, CA (US)

(21) Appl. No.: 16/714,415

(22) Filed: Dec. 13, 2019

Related U.S. Application Data
(63) Continuation of application No. PCT/US2018/036936, filed on Jun. 11, 2018.
(60) Provisional application No. 62/522,292, filed on Jun. 20, 2017.

(51) Int. Cl. B01L 3/00; C12N 15/10; C08G 81/00; B01F 3/08; B01F 13/00; B01F 17/00; G01N 15/10

(52) U.S. Cl. CPC B01L 3/502784; C12N 15/10; C08G 81/00; B01F 3/0807; B01F 13/0062; B01F 17/0092; G01N 2015/1006; G01N 15/10; B01L 2200/0673; B01L 2300/0816; B01L 2300/0867; B01F 2215/0037; B01F 17/0028

(57) ABSTRACT

The present disclosure provides methods for forming emulsions. A method for forming an emulsion comprises: bringing a first fluid phase in contact with a second fluid phase that is immiscible with the first fluid phase to generate the emulsion comprising the plurality of droplets. The plurality of droplets may comprise (i) the first fluid phase or the second fluid phase, (ii) a first surfactant at an interface between the first fluid phase and the second fluid phase, and (iii) a second surfactant that is different than the first surfactant. Subsequent to generating the emulsion, collect or direct the plurality of droplets along a channel. Subsequent to collecting or directing the plurality of droplets along the channel, at most 5% of the plurality of droplets coalesce.

### CROSS REFFERENCE

This application is a continuation of International Patent Application No. PCT/US2018/036936, filed Jun. 11, 2018, which claims priority to U.S. Provisional Patent Application No. 62/522,292, filed Jun. 20, 2017, which are entirely incorporated herein by reference for all purposes.

### BACKGROUND

Significant advances in materials and systems to analyze and characterize biological and biochemical materials have led to advances in understanding the mechanisms of life, health, disease and treatment. In particular, genomic sequencing can be used to obtain biomedical information in diagnostics, prognostics, biotechnology, and forensics. Specifically, many deoxyribonucleic acid (DNA) sequencing technologies involve the segmentation and processing of genomic materials into manageable sized barcoded fragments.

One method often used in sequencing technologies is to analyze genomic materials through the partitioned analysis of contents of sample nucleic acids or cells. In this approach, an individual sample nucleic acid or a cell is co-partitioned with processing reagents, typically in emulsion droplets, before sequencing steps. One configuration of emulsion comprises an aqueous phase and a hydrocarbon oil phase. However, many challenges still remain unaddressed, or not fully addressed by the current solutions.

### SUMMARY

As recognized herein, emulsions may comprise a hydrocarbon oil phase and an aqueous phase. Fluorocarbon oils may be immiscible with both the hydrocarbon oils and the aqueous phase. As a result, either hydrocarbon oils or the aqueous phase may be dispersed as emulsion droplets in a fluorocarbon phase which can be the continuous phase of an emulsion. Stabilization of emulsions in fluorocarbon oil phase may require the addition of appropriate surfactants to form the barrier between the fluorocarbon phase and the aqueous phase/hydrocarbon phase. Accordingly, surfactant systems for stabilizing droplets of water and hydrocarbon oil or organic solvents in a continuous fluorophilic phase are needed.

Provided herein are methods, systems and compositions for the stabilization of aqueous emulsions formed in fluorocarbon oil continuous phase. A fluorosurfactant having one fluorophilic tail and one hydrophilic head group (hereinafter “di-block surfactant” or “di-block copolymer”) may be used to reduce the coalescence of emulsion droplets, including, for example, gel bead-in-emulsion systems. This di-block surfactant may provide better stability for emulsion systems than those with other fluorosurfactants, including fluorosurfactants having two fluorophilic tails and one hydrophilic head group (hereinafter “tri-block surfactant”).

In an aspect, the present disclosure provides a method for forming an emulsion comprising a plurality of droplets, comprising: (a) bringing a first fluid phase in contact with a second fluid phase that is immiscible with the first fluid phase to generate an emulsion comprising the plurality of droplets, wherein the plurality of droplets comprises (i) the first fluid phase or the second fluid phase, (ii) a first surfactant at an interface between the first fluid phase and the second fluid phase, and (iii) a second surfactant that is different than the first surfactant; and (b) upon generating at least a subset of the plurality of droplets, (i) collecting the plurality of droplets or (ii) directing the plurality of droplets along a channel, wherein upon collecting or directing the plurality of droplets along the channel, at most 5% of the plurality of droplets coalesce.

In some embodiments of aspects provided herein, the first surfactant at the interface prevents the second surfactant from flowing from the first fluid phase to the second fluid phase. In some embodiments of aspects provided herein, the first surfactant is a di-block copolymer comprising a perfluorinated polyether block bonded to a polyethylene glycol block. In some embodiments of aspects provided herein, the di-block copolymer reduces droplet coalescence when compared with a tri-block copolymer comprising at least two perfluorinated polyether blocks bonded to a polyethylene glycol block. In some embodiments of aspects provided herein, at least a subset of the droplet coalescence is surface-mediated. In some embodiments of aspects provided herein, the di-block copolymer reduces the surface-mediated droplet coalescence when compared with the tri-block copolymer. In some embodiments of aspects provided herein, the di-block copolymer is a compound of Formula II:

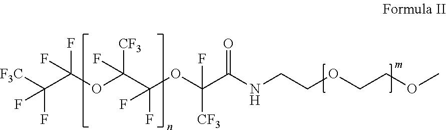

wherein m is an integer from 5 to 50 and n is an integer from 5 to 60. In some embodiments of aspects provided herein, the di-block copolymer is at a concentration from about 2.5 mM to about 3.0 mM. In some embodiments of aspects provided herein, the second surfactant is n-dodecyl-D-maltoside In some embodiments of aspects provided herein, the second surfactant facilitates cell lysis. In some embodiments of aspects provided herein, the plurality of droplets comprise reagents necessary for nucleic acid amplification. In some embodiments of aspects provided herein, the plurality of droplets comprises particles with nucleic acid barcodes. In some embodiments of aspects provided herein, the particles are gel beads. In some embodiments of aspects provided herein, an individual droplet of the plurality of droplets comprises at most one particle from the particles. In some embodiments of aspects provided herein, the first fluid phase is an aqueous phase and the second fluid phase is a non-aqueous phase. In some embodiments of aspects provided herein, the non-aqueous phase is an oil phase. In some embodiments of aspects provided herein, the non-aqueous phase comprises a fluorinated oil. In some embodiments of aspects provided herein, the plurality of droplets comprises biological molecules. In some embodiments of aspects provided herein, the biological molecules comprise nucleic acid molecules. In some embodiments of aspects provided herein, at most 2% of the plurality of droplets coalesce. In some embodiments of aspects provided herein, the plurality of droplets is generated at an intersection of at least a first channel and a second channel, wherein the first fluid phase or the second fluid phase, but not both, is directed along the first channel.

Another aspect of the present disclosure provides a system for forming an emulsion comprising a plurality of droplets, comprising a droplet generator that is configured to generate an emulsion comprising a plurality of droplets; and a controller operatively coupled to the droplet generator, wherein the controller is programed to: (i) bring a first fluid phase in contact with a second fluid phase that is immiscible with the first fluid phase to generate the emulsion comprising the plurality of droplets, wherein the plurality of droplets comprises (1) the first fluid phase or the second fluid phase, (2) a first surfactant at an interface between the first fluid phase and the second fluid phase, and (3) a second surfactant that is different than the first surfactant; and (ii) upon generating at least a subset of the plurality of droplets, (i) direct collection of the plurality of droplets or (ii) direct the plurality of droplets along a channel, wherein upon collecting or directing the plurality of droplets along the channel, at most 5% of the plurality of droplets coalesce.

Still another aspect of the present disclosure provides a non-transitory computer-readable medium comprising machine-executable code that, upon execution by one or more computer processors, implements method for forming an emulsion comprising a plurality of droplets, the method comprising: (a) bringing a first fluid phase in contact with a second fluid phase that is immiscible with the first fluid phase to generate an emulsion comprising the plurality of droplets, wherein the plurality of droplets comprises (i) the first fluid phase or the second fluid phase, (ii) a first surfactant at an interface between the first fluid phase and the second fluid phase, and (iii) a second surfactant that is different than the first surfactant; and (b) upon generating at least a subset of the plurality of droplets, (i) collecting the plurality of droplets or (ii) directing the plurality of droplets along a channel, wherein upon collecting or directing the plurality of droplets along the channel, at most 5% of the plurality of droplets coalesce.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG” and “FIGs” herein), of which:

FIG. 1 schematically illustrates a droplet generator comprising microfluidic channel structure for partitioning individual or small groups of cells.

FIG. 2 schematically illustrates a droplet generator comprising a microfluidic channel structure for co-partitioning cells and microcapsules (e.g., beads) comprising additional reagents.

FIG. 3A schematically illustrates an overview of an example process for preparation of barcoded sequencing samples.

FIG. 3B schematically illustrates an operation in a process for preparation of barcoded sequencing samples.

FIG. 3C schematically illustrates another operation in a process for preparation of barcoded sequencing samples.

FIG. 4 depicts a picture of pipettes containing coalesced emulsion droplets.

FIG. 5 demonstrates pictures of coalesced emulsion droplets in wells and the roughness of the corresponding wells.

FIG. 6 shows an example of tri-block surfactant according to Formula I.

FIG. 7 illustrates an example of di-block surfactant according to Formula II.

FIG. 8 depicts pictures of pipettes using formulations containing tri-block surfactant and di-block surfactant, respectively, according to the present disclosure.

FIG. 9 schematically illustrates a diagram to explain the observed reduced coalescence in formulation comprising a two-block surfactant.

FIG. 10 shows an example computer control system that is programmed or otherwise configured to implement methods provided herein.

FIG. 11 provides pictures of emulsions made from formulations containing the di-block surfactant at different concentrations.

### DETAILED DESCRIPTION

While various embodiments of the invention have been shown and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

As used in this specification and the appended claims, the singular forms “a,” “an” and “the” include plural referents unless the context clearly dictates otherwise. Thus, for example, reference to “a molecule” includes a plurality of such molecules, and the like.

The term “sample,” as used herein, generally refers to a biological sample of a subject. The biological sample may be a nucleic acid sample or protein sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swap. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

As used herein, the term “barcode” generally refers to a known, determinable or decodable sequence, such as a nucleic acid sequence, that allows the identification of some feature of a biological sample, such as a nucleic acid (e.g., oligonucleic acids), with which the barcode is associated. The barcodes can be designed for precision sequence performance, e.g., GC content between 40% and 60%, no homo-polymer runs longer than two, no self-complementary stretches longer than 3, and be comprised of sequences not present in a human genome reference. Barcodes can be of sufficient length and comprise sequences that can be sufficiently different to allow the identification of each nucleic acid (e.g., oligonucleic acids) based on barcode(s) with which each nucleic acid is associated. Further, as used herein, the reference to barcode sequences also includes the complements to any such barcode sequences.

The term “target nucleic acid” as used herein generally refers to the nucleic acid or nucleic acid fragment targeted for detection and/or sequencing analysis. Sources of target nucleic acids may be isolated from organisms, including mammals, or pathogens to be identified, including viruses and bacteria. Additionally target nucleic acids can also be from synthetic sources. Target nucleic acids may be or may not be amplified via standard replication/amplification procedures to produce nucleic acid sequences.

The term “nucleic acid sequence” or “nucleotide sequence” as used herein generally refers to nucleic acid molecules with a given sequence of nucleotides, of which it may be desired to know the presence or amount. The nucleotide sequence can comprise ribonucleic acid (RNA) or DNA, or a sequence derived from ribonucleic acid (RNA) or deoxyribonucleic acid (DNA). Examples of nucleotide sequences are sequences corresponding to natural or synthetic RNA or DNA including genomic DNA and messenger RNA. The length of the sequence can be any length that can be amplified into nucleic acid amplification products, or amplicons, for example, up to about 5, 10, 20, 30, 40, 50, 100, 200, 300, 400, 500, 600, 700, 800, 1,000, 1,200, 1,500, 2,000, 5,000, 10,000 or more than 10,000 nucleotides in length, or at least about 5, 10, 20, 30, 40, 50, 100, 200, 300, 400, 500, 600, 700, 800, 1,000, 1,200, 1,500, 2,000, 5,000, 10,000, 10,000, or greater nucleotides in length.

The term “template” as used herein generally refers to individual polynucleotide molecules from which another nucleic acid, including a complementary nucleic acid strand, can be synthesized by a nucleic acid polymerase. In addition, the template can be one or both strands of the polynucleotides that are capable of acting as templates for template-dependent nucleic acid polymerization catalyzed by the nucleic acid polymerase. Use of this term should not be taken as limiting the scope of the present disclosure to polynucleotides which are actually used as templates in a subsequent enzyme-catalyzed polymerization reaction.

As used herein, the terms “adaptor(s)”, “adapter(s)” and “tag(s)” generally refer to a polynucleotide sequence used to either attach single polynucleotide fragments to beads and/or to prime the emulsion PCR reaction and/or as a template to prime pyrosequencing reactions. An adaptor or tag can be coupled to another polynucleotide sequence by approaches including ligation, hybridization, or other approaches.

The term “bead,” as used herein, generally refers to a particle. The bead may be a solid or semi-solid particle. The bead may be a gel. The bead may be formed of a polymeric material. The bead may be magnetic or non-magnetic.

As used herein, the term “emulsion” generally refers to a stable mixture of at least two immiscible liquids. Immiscible liquids may tend to separate into two distinct phases. An emulsion may be stabilized by the addition of a “surfactant” which may reduce the surface tension between the two immiscible liquids and/or to stabilize the interface therebetween. In some cases, emulsion described herein may include a discontinuous or disperse phase (i.e., the isolated phase stabilized by a surfactant) formed of an aqueous or lipophilic (e.g., hydrocarbon) substance. The continuous phase may be formed of a fluorophilic substance (e.g., a fluorocarbon). The present disclosure may involve, in some cases, water-in-fluorocarbon emulsions and hydrocarbon-in-fluorocarbon emulsions having a disperse aqueous or hydrocarbon phase and a fluorocarbon continuous phase. The isolated disperse aqueous or lipophilic phase in a fluorophilic solvent can form a “reverse emulsion,” which is simply one example of an emulsion. In some cases, the emulsions described herein are macroemulsions. Macroemulsions are emulsions that are kinetically stable, as compared to microemulsions, which are thermodynamically stable and undergo spontaneous formation. In some cases, a microemulsion may include droplets having an average diameter of less than 50 nm.

As used herein, the term “droplet” generally refers to an isolated aqueous or lipophilic phase within a continuous phase having any shapes, including, for example cylindrical, spherical, ellipsoidal, irregular shapes, etc. Generally, in emulsions of the present disclosure, aqueous and/or lipophilic droplets are spherical or substantially spherical in a fluorocarbon, continuous phase.

As used herein, the term “surfactant” generally refers to a molecule that, when combined with a first component defining a first phase, and a second component defining a second phase, will facilitate assembly of separate first and second phases. In some cases, a surfactant of present disclosure may have one or more main fluorophilic chain(s) where one end of the chain is soluble in the fluorophilic phase of the emulsion, and one or more chains that are not soluble in the fluorophilic phase of the emulsion (e.g., those chains may be soluble in the aqueous or lipophilic phase).

As used herein, the term “fluorophilic” generally refers to a component comprising any fluorinated compound, including, for example, as a linear, branched, cyclic, saturated, or unsaturated fluorinated hydrocarbon. The fluorophilic component may optionally include at least one heteroatom (e.g., in the backbone of the component). In some cases, the fluorophilic compound may be fluorinated, i.e., at least 30%, at least 50%, at least 70%, or at least 90% of the hydrogen atoms of the component are replaced by fluorine atoms. The fluorophilic component may comprise a fluorine to hydrogen ratio of, for example, at least 0.2:1, at least 0.5:1, at least 1:1, at least 2:1, at least 5:1, or at least 10:1. In some cases, at least 30%, at least 50%, at least 70%, or at least 90% but less than 100% of the hydrogen atoms of the component is replaced by fluorine atoms. In other cases, the fluorophilic component is perfluorinated, i.e., the component contains fluorine atoms but contains no hydrogen atoms. Fluorophilic components compatible with the present disclosure may have low toxicity, low surface tension, and the ability to dissolve and transport gases.

As used herein, the term “nonaqueous” generally refers to materials such as a fluid that is immiscible with water. That is, a liquid that when mixed with water will form a stable two-phase mixture. The non-aqueous phase need not be liquid, but can be a solid or semi-solid lipid or other nonpolar substance that is not soluble in water. In some instances, the nonaqueous phase can include a lipophilic component (e.g., a hydrocarbon) or a fluorinated component (e.g., a fluorocarbon). The aqueous phase can be any liquid miscible with water; that is, any liquid that, when admixed with water, can form a room-temperature, single-phase solution that is stable. In some cases, the aqueous phase can comprise one or more physiologically acceptable reagents and/or solvents, etc. Non-limiting examples of aqueous phase materials may include (besides water itself) methanol, ethanol, DMF (dimethylformamide), or DMSO (dimethyl sulfoxide).

As used herein, the terms “coalescence” and “coalesce” generally refer to the phenomenon in emulsion systems when one droplet is paired and merged with at least one other droplet so that a bigger droplet is produced in the end.

As used herein, “amplification” of a template nucleic acid generally refers to a process of creating (e.g., in vitro) nucleic acid strands that are identical or complementary to at least a portion of a template nucleic acid sequence, or a universal or tag sequence that serves as a surrogate for the template nucleic acid sequence, all of which are only made if the template nucleic acid is present in a sample. Typically, nucleic acid amplification uses one or more nucleic acid polymerase and/or transcriptase enzymes to produce multiple copies of a template nucleic acid or fragments thereof, or of a sequence complementary to the template nucleic acid or fragments thereof. In vitro nucleic acid amplification techniques are may include transcription-associated amplification methods, such as Transcription-Mediated Amplification (TMA) or Nucleic Acid Sequence-Based Amplification (NASBA), and other methods such as Polymerase Chain Reaction (PCR), Reverse Transcriptase-PCR (RT-PCR), Replicase Mediated Amplification, and Ligase Chain Reaction (LCR).

As used herein, the term “isothermal amplification” generally refers to an amplification reaction that is conducted at a substantially constant temperature. The isothermal portion of the reaction may be preceded or followed by one or more operations at a variable temperature, for example, a first denaturation step and a final heat inactivation step or cooling step. It will be understood that this definition does not exclude certain, in some cases small, variations in temperature but is rather used to differentiate the isothermal amplification techniques from other amplification techniques that may rely on “cycling temperatures” in order to generate the amplified products. Isothermal amplification differs from PCR, for example, in that the latter relies on cycles of denaturation by heating followed by primer hybridization and polymerization at a lower temperature. Isothermal amplification can rely on chemistries, including but not limited to, loop-mediated isothermal amplification (LAMP), strand displacement amplification (SDA), helicase-dependent amplification (HDA), and nicking enzyme amplification reaction (NEAR).

Sequence information of nucleic acids may be the foundation to improve people's lives through clinical approaches or by material approaches. (See, Ansorge, W., “Next-generation DNA sequencing techniques,” New Biotech. (2009) 25(4):195-203, which is entirely incorporated herein by reference). Several parallel DNA sequencing platforms have been available on the market. The availability of NGS accelerates biological and biomedical research enables the comprehensive analysis of genomes, transcriptomes and interactomes. (See, Shendure, J. and Ji, H., “Next-generation DNA sequencing,” Nature Biotech. (2008) 26:1135-45, which is entirely incorporated herein by reference). One particular challenge faced by researchers in the NGS filed is a more robust protocol for generating sequencing samples, for example, barcoded samples.

Commonly used and commercially available NGS sequencing platforms include the Illumina Genome Analyzer, the Roche (454) Genome Sequencer, the Life Technologies SOLiD platform, and real-time sequencers such as Pacific Biosciences. Nucleic acid sequencing technologies may derive the nucleic acids that they sequence from collections of cells obtained from tissue or other samples, such as biological fluids (e.g., blood, plasma, etc). The cells can be processed (e.g., all together) to extract the genetic material that represents an average of the population of cells, which can then be processed into sequencing ready DNA samples that are configured for a given sequencing technology. Although often discussed in terms of DNA or nucleic acids, the nucleic acids derived from the cells may include DNA, or RNA, including, e.g., mRNA, total RNA, or the like, that may be processed to produce cDNA for sequencing. Following processing, absent a cell specific marker, attribution of genetic material as being contributed by a subset of cells or an individual cell may not be possible in such an ensemble approach.

Therefore, there is a need for characterizing nucleic acids from small populations of cells, and in some cases for characterizing nucleic acids from individual cells, especially in the context of larger population of cells.

### Compartmentalization and Characterization of Cells

The present disclosure provides methods for stabilizing emulsion droplets usable in characterization of nucleic acids from small populations of cells, and in some cases, from individual cells, especially in the context of larger populations of cells. Disclosed herein are methods and systems for characterizing surface features, proteins, and nucleic acids of small populations of cells, and in some cases, for characterizing surface features, proteins, and nucleic acids of individual cells. The methods described herein may compartmentalize the analysis of individual cells or small populations of cells, including e.g., cell surface features, proteins, and nucleic acids of individual cells or small groups of cells, and then allow that analysis to be attributed back to the individual cell or small group of cells from which the cell surface features, proteins, and nucleic acids are derived. This can be accomplished regardless of whether the cell population represents a 50/50 mix of cell types, a 90/10 mix of cell types, or virtually any ratio of cell types, as well as a complete heterogeneous mix of different cell types, or any mixture between these.

The stabilized emulsion droplets can be used in construction of sequencing samples. These sequencing samples produced according to the present disclosure can provide sequencing results, for example, whole genome sequencing results, when coupled with sequencing methods or systems. The sequencing samples produced according to the present disclosure can be employed in nucleic acid analysis applications, such as, for example, nucleic acid sequencing applications.

A method often used construct a set of DNA samples is called emulsion PCR (E-PCR) with microbeads. E-PCR method is used by Roche's 454 (Margulies, et al., “Genome Sequencing in Microfabricated High-density Picolitre Reactors,” Nature (2005) 437(7057):376-80) and Life Technologies' SOLiD (Valouev, et al., “A High-resolution, Mucleosome Position Map of C. Elegans Reveals a Lack of Universal Sequence-dictated Positioning,” Genome Res. (2008) 18(7):1051-63) and Ion Torrent (Rothberg, et al., “An Integrated Semiconductor Device Enabling Non-optical Genome Sequencing,” Nature (2011) 475(7356):348-52) platforms, all of which are entirely incorporated herein by reference. E-PCR can require performing PCR on billions of microbeads, each isolated in its own emulsion droplet, followed by emulsion breakup, template enrichment, and bead deposition before sequencing. The methods and systems disclosed in the present disclosure can be applicable in E-PCR.

This disclosure also provides methods, systems and compositions useful in the processing of sample materials, for example, nucleic acids samples, through the controlled delivery of reagents to subsets of sample components, followed by analysis of those sample components employing, in part, the delivered reagents. In many cases, the methods and compositions can be employed for sample processing, particularly for nucleic acid analysis applications, generally, and nucleic acid sequencing applications, in particular. Included within this disclosure are bead compositions that include diverse sets of reagents, such as diverse samples of beads attached to large numbers of oligonucleotides containing barcode sequences, and methods of making and using the same. Methods, systems and composition, described in U.S. Patent Publication Nos. 2015/0376609 and 2016/0257984, each of which is entirely incorporated herein by reference, may process samples materials, including nucleic acids samples, by using a set of beads with oligonucleotide barcodes.

The methods, systems and composition of this present disclosure may be used with bead or particle, including, for example, gel beads and other types of beads. Beads may serve as a carrier for reagents that are to be delivered in accordance with the methods described herein. In some cases, these beads may provide a surface to which reagents are releasably attached, or a volume in which reagents are entrained or otherwise releasably partitioned. These reagents may then be delivered in accordance with methods described herein, for example, in the controlled delivery of reagents into discrete partitions. A variety of different reagents or reagent types may be associated with the beads, when delivering such reagents to a partition. Non-limiting examples of such reagents delivered include, e.g., enzymes, polypeptides, antibodies or antibody fragments, labeling reagents, e.g., dyes, fluorophores, chromophores, etc., nucleic acids, polynucleotides, oligonucleotides, and any combination of two or more of the foregoing. In some cases, the beads may provide a surface upon which to synthesize or attach oligonucleotide sequences. Various entities including oligonucleotides, barcode sequences, primers, adaptors, linkers, and/or cross-linkers may be associated with the outer surface of a bead. In the case of porous beads, an entity may be associated with both the outer and inner surfaces of a bead. The entities may be attached directly to the surface of a bead (e.g., via a covalent bond, ionic bond, van der Waals interactions, etc.), may be attached to other oligonucleotide sequences attached to the surface of a bead (e.g. adaptor or primers), may be diffused throughout the interior of a bead and/or may be combined with a bead in a partition (e.g. fluidic droplet). In some cases, the oligonucleotides can be covalently attached to sites within the polymeric matrix of the bead and are therefore present within the interior and exterior of the bead. In some cases, an entity such as a cell or nucleic acid may be encapsulated within a bead. Other entities including amplification reagents (e.g., PCR reagents, primers) may also be diffused throughout the bead or chemically-linked within the interior (e.g., via pores, covalent attachment to polymeric matrix) of a bead.

Beads may serve to localize entities or samples. In some cases, entities (e.g. oligonucleotides, barcode sequences, primers, cross-linkers, adaptors and the like) may be associated with the outer and/or an inner surface of the bead. In some cases, entities may be located throughout the bead. In some cases, the entities may be associated with the entire surface of a bead or with at least half the surface of the bead.

Beads may serve as a support on which to synthesize oligonucleotide sequences. In some cases, synthesis of an oligonucleotide may comprise a ligation step. In some cases, synthesis of an oligonucleotide may comprise ligating two smaller oligonucleotides together. In some cases, a primer extension or other amplification reaction may be used to synthesize an oligonucleotide on a bead via a primer attached to the bead. In such cases, a primer attached to the bead may hybridize to a primer binding site of an oligonucleotide that also contains a template nucleotide sequence. The primer can then be extended by a primer extension reaction or other amplification reaction, and an oligonucleotide complementary to the template oligonucleotide can thereby be attached to the bead. In some cases, a set of identical oligonucleotides associated with a bead may be ligated to a set of diverse oligonucleotides, such that each identical oligonucleotide is attached to a different member of the diverse set of oligonucleotides. In some cases, a set of diverse oligonucleotides associated with a bead may be ligated to a set of identical oligonucleotides. In some cases, the set of diverse oligonucleotides may be a set of fragments of a target nucleic acid. In some cases, the set of identical oligonucleotides may be adaptors or nucleic acids comprising barcodes.

Methods of making beads can generally include, for example, combining bead precursors (such as monomers or polymers), primers or adaptors, and cross-linkers in an aqueous solution, combining the aqueous solution with an oil phase, sometimes using a microfluidic device or droplet generator, and causing water-in-oil droplets to form.

In some cases, a catalyst, such as an accelerator and/or an initiator, can be added before or after droplet formation. In some cases, initiation can be achieved by the addition of energy, such as, for example, via the addition of heat or light (e.g., UV light). A polymerization reaction of bead precursors in the droplet can occur to generate a bead.

In some cases, the bead can be covalently linked to one or more copies of an oligonucleotide (e.g., primer or adaptor) to become functionalized. Additional nucleic acid sequences can be attached to the functionalized beads using a variety of methods. In some cases, the functionalized beads may be combined with a template oligonucleotide (e.g., a barcode) and partitioned such that on average one or fewer template oligonucleotides may occupy the same partition as a functionalized bead. While the partitions can be any of a variety of different types of partitions, e.g., wells, microwells, tubes, vials, microcapsules, etc., in some cases, the partitions can be droplets (e.g., aqueous droplets) within an emulsion.

Beads may be made in a device or beads (or other types of partitions) may be combined in a device with samples, e.g., for co-partitioning sample components. The device may be a microfluidic device (e.g., a droplet generator). In some cases, the device may be formed from a material selected from the group consisting of fused silica, soda lime glass, borosilicate glass, poly (methyl methacrylate) PMMA, PDMS, sapphire, silicon, germanium, cyclic olefin copolymer, polyethylene, polypropylene, polyacrylate, polycarbonate, plastic, thermosets, hydrogels, thermoplastics, paper, elastomers, and combinations thereof.

The device may comprise fluidic channels for the flow of fluids. In some cases, a device may comprise one or more fluidic input channels (e.g., inlet channels) and one or more fluidic outlet channels. In some cases, the microfluidic device may be utilized to form beads by forming a fluidic droplet comprising one or more gel precursors, one or more cross-linkers, optionally an initiator, and optionally an aqueous surfactant.

The microfluidic device may be used to combine beads (e.g., barcoded beads or other type of first partition) with sample (e.g., a sample of nucleic acids) by forming a fluidic droplet (or other type of second partition) comprising both the beads and the sample. The fluidic droplet may have an aqueous core surrounded by an oil phase, such as, for example, aqueous droplets within a water-in-oil emulsion. The oil may further comprise a surfactant and/or an accelerator. The fluidic droplet may contain one or more barcoded beads, a sample, amplification reagents, and a reducing agent. In some cases, the fluidic droplet may include one or more of water, nuclease-free water, acetonitrile, beads, gel beads, polymer precursors, polymer monomers, polyacrylamide monomers, acrylamide monomers, degradable cross-linkers, non-degradable cross-linkers, disulfide linkages, acrydite moieties, PCR reagents, primers, polymerases, barcodes, polynucleotides, oligonucleotides, nucleotides, DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, probes, dyes, organics, emulsifiers, surfactants, stabilizers, polymers, aptamers, reducing agents, initiators, biotin labels, fluorophores, buffers, acidic solutions, basic solutions, light-sensitive enzymes, pH-sensitive enzymes, aqueous buffer, oils, salts, detergents, ionic detergents, non-ionic detergents, and the like. The composition of the fluidic droplet may vary depending on the particular processing needs. The fluidic droplets may be of uniform size or heterogeneous size.

The device may comprise one or more intersections of two or more fluid input channels. For example, the intersection may be a fluidic cross. The fluidic cross may comprise two or more fluidic input channels and one or more fluidic outlet channels. In some cases, the fluidic cross may comprise two fluidic input channels and two fluidic outlet channels. In some cases, the fluidic cross may comprise three fluidic input channels and one fluidic outlet channel. In some cases, the fluidic cross may form a substantially perpendicular angle between two or more of the fluidic channels forming the cross.

A microfluidic device may comprise a first and second input channels that meet at a junction that is fluidly connected to an output channel. In some cases, the output channel may be, for example, fluidly connected to a third input channel at another junction. In some cases, a fourth input channel may be included and may intersect the third input channel and the outlet channel at still another junction. In some cases, a microfluidic device may comprise first, second, and third input channels, wherein the third input channel may intersect the first input channel, the second input channel, or a junction of the first input channel and the second input channel.

The microfluidic device may be used to generate gel beads from a liquid. For example, in some cases, an aqueous fluid comprising one or more gel precursors, one or more cross-linkers and optionally an initiator, optionally an aqueous surfactant, and optionally an alcohol within a fluidic input channel may enter a fluidic cross. Within a second fluidic input channel, an oil with optionally a surfactant and an accelerator may enter the same fluidic cross. Both aqueous and oil components may be mixed at the fluidic cross to form aqueous fluidic droplets within the continuous oil phase. Gel precursors within fluidic droplets exiting the fluidic cross may polymerize to form beads.

The microfluidic device may be used to combine sample with beads (e.g., a set of barcoded beads) as well as an agent capable of degrading the beads (e.g., reducing agent if the beads are linked with disulfide bonds). In some cases, a sample (e.g., a sample of nucleic acids) may be provided to a first fluidic input channel that is fluidly connected to a first fluidic cross (e.g., a first fluidic junction). Pre-formed beads (e.g., barcoded beads, degradable barcoded beads) may be provided to a second fluidic input channel that is also fluidly connected to the first fluidic cross, where the first fluidic input channel and second fluidic input channel meet. The sample and beads may be mixed at the first fluidic cross to form a new mixture (e.g., an aqueous mixture). In some cases, a reducing agent may be provided to a third fluidic input channel that is also fluidly connected to the first fluidic cross and meets the first and second fluidic input channels at the first fluidic cross. The reducing agent can then be mixed with the beads and the sample in the first fluidic cross. In some cases, the reducing agent may be premixed with the sample and/or the beads before entering the microfluidic device such that it is provided to the microfluidic device through the first fluidic input channel with the sample and/or through the second fluidic input channel with the beads. In some cases, no reducing agent may be added.

The sample and bead mixture may exit the first fluidic cross through a first outlet channel that is fluidly connected to the first fluidic cross (and, thus, any fluidic channels forming the first fluidic cross). The mixture may be provided to a second fluidic cross (e.g., a second fluidic junction) that is fluidly connected to the first outlet channel. In some cases, an oil (or other suitable immiscible) fluid may enter the second fluidic cross from one or more separate fluidic input channels that are fluidly connected to the second fluidic cross (and, thus, any fluidic channels forming the cross) and that meet the first outlet channel at the second fluidic cross. In some cases, the oil (or other suitable immiscible fluid) may be provided in one or two separate fluidic input channels fluidly connected to the second fluidic cross (and, thus, the first outlet channel) that meet the first outlet channel and each other at the second fluidic cross. The oil, and the sample and bead mixture, may be mixed at the second fluidic cross. This mixing may partition the sample and bead mixture into a plurality of fluidic droplets (e.g., aqueous droplets within a water-in-oil emulsion), in which at least a subset of the droplets may encapsulate a barcoded bead (e.g., a gel bead). The fluidic droplets that formed may be carried within the oil through a second fluidic outlet channel exiting from the second fluidic cross. In some cases, fluidic droplets exiting the second outlet channel from the second fluidic cross may be partitioned into wells for further processing.

In many cases, it may be desirable to control the occupancy rate of resulting droplets (or second partitions) with respect to beads (or first partitions). An example of such control is described in U.S. Patent Publication No. 2015/0292988, which is entirely incorporated herein by reference. In general, the droplets (or second partitions) can be formed such that at least 50%, 60%, 70%, 80%, 90% or more droplets (or second partitions) contain no more than one bead (or first partition). Additionally, or alternatively, the droplets (or second partitions) can be formed such that at least 50%, 60%, 70%, 80%, 90% or more droplets (or second partitions) include exactly one bead (or first partition). In some cases, the resulting droplets (or second partitions) may each comprise, on average, at most about one, two, three, four, five, six, seven, eight, nine, ten, eleven, twelve, thirteen, fourteen, fifteen, sixteen, seventeen, eighteen, nineteen, or twenty beads (or first partitions). In some cases, the resulting droplets (or second partitions) may each comprise, on average, at least about one, two, three, four, five, six, seven, eight, nine, ten, eleven, twelve, thirteen, fourteen, fifteen, sixteen, seventeen, eighteen, nineteen, twenty, or more beads (or first partitions).

The methods, compositions, and devices of the present disclosure may be used with many suitable oils. In some cases, an oil may be used to generate an emulsion. The oil may comprise fluorinated oil, silicon oil, mineral oil, vegetable oil, and combinations thereof.

Any suitable number of nucleic acid molecules (e.g., primer, barcoded oligonucleotide, anchor oligonucleotide) can be associated with a bead such that, upon release from the bead, the nucleic acid molecules (e.g., primer, barcoded oligonucleotide, and anchor oligonucleotide) are present in the partition at a pre-defined concentration. Such pre-defined concentration may be selected to facilitate certain reactions for generating a set of sequencing samples, e.g., amplification, within the partition. In some cases, the pre-defined concentration of the primer is limited by the process of producing oligonucleotide bearing beads.

The template oligonucleotide (e.g., containing barcode) sequences can be attached to the beads within the partition by a reaction such as a primer extension reaction, ligation reaction, or other methods. For example, in some cases, beads functionalized with primers can be combined with template barcode oligonucleotides that comprise a binding site for the primer, enabling the primer to be extended on the bead. After multiple rounds of amplification, copies of the single barcode sequence can be attached to the multiple primers attached to the bead. After attachment of the barcode sequences to the beads, the emulsion can be broken and the barcoded beads (or beads linked to another type of amplified product) can be separated from beads without amplified barcodes. Additional sequences, such as a random sequence (e.g., a random N-mer) or a nucleic acid target sequence, can then be added to the bead-bound barcode sequences, using, for example, primer extension methods or other amplification reactions. This process can generate a large and diverse set of barcoded beads.

Barcodes can be generated from a variety of different formats, including bulk synthesized polynucleotide barcodes, randomly synthesized barcode sequences, microarray based barcode synthesis, native nucleotides, partial complement with N-mer, random N-mer, pseudo random N-mer, or combinations thereof. Synthesis of barcodes is described herein, as well as in, for example, in U.S. Patent Publication No. 2014/0228255, which is entirely incorporated herein by reference.

The barcodes may be loaded into beads so that one or more barcodes are introduced into a particular bead. In some cases, each bead may contain the same set of barcodes. In some cases, each bead may contain different sets of barcodes. In some cases, each bead may comprise a set of identical barcodes. In some cases, each bead may comprise a set of different barcodes.

Template oligonucleotide can incorporate additional sequence segments other than barcode sequence segments. Such additional sequence segments can include functional sequences, such as primer sequences, and primer annealing site sequences. In addition, functional sequences can include, for example, immobilization sequences for immobilizing barcode containing sequences onto surfaces, e.g., for sequencing applications. For ease of discussion, a number of specific functional sequences are described below, such as primers of P5, P7, Read1primer, and Read2primer (or others), sample indexes, random N-mers, etc., and partial sequences for these, as well as complements of any of the foregoing. However, it will be appreciated that these descriptions are for purposes of discussion, and any of the various functional sequences included within the barcode containing oligonucleotides can be substituted for these specific sequences, including without limitation, different attachment sequences, different sequencing primer regions, different N-mer regions (targeted and random), as well as sequences having different functions, e.g., secondary structure forming, e.g., hairpins or other structures, probe sequences, e.g., to allow interrogation of the presence or absence of the oligonucleotides or to allow pull down of resulting amplicons, or any of a variety of other functional sequences.

Also included within this disclosure are methods of sample preparation for nucleic acid analysis, and particularly for sequencing applications. Sample preparation can generally include, e.g., obtaining a sample comprising sample nucleic acid from a source, optionally further processing the sample, combining the sample nucleic acid with barcoded beads, and forming emulsions containing fluidic droplets comprising the sample nucleic acid and the barcoded beads. Droplets can be generated, for example, with the aid of a microfluidic device and/or via any suitable emulsification method. The fluidic droplets can also comprise agents capable of dissolving, degrading, or otherwise disrupting the barcoded beads, and/or disrupting the linkage to attached sequences, thereby releasing the attached barcode sequences from the bead. The barcode sequences can be released either by degrading the bead, detaching the oligonucleotides from the bead such as by a cleavage reaction, or a combination of both.

By amplifying (e.g., via amplification methods described herein) the sample nucleic acid in the fluidic droplets, the free barcode sequences can be attached to the sample nucleic acid. The emulsion comprising the fluidic droplets can then be broken and, if desired, additional sequences (e.g., sequences that aid in particular sequencing methods, additional barcode sequences, etc.) can then be added to the barcoded sample nucleic acid using, for example, additional amplification methods. Sequencing can then be performed on the barcoded, amplified sample nucleic acid and one or more sequencing algorithms applied to interpret the sequencing data. As used herein, the sample nucleic acids can include any of a wide variety of nucleic acids, including, e.g., DNA and RNA, and specifically including for example, genomic DNA, cDNA, mRNA total RNA, and cDNA created from mRNA or total RNA transcript.

The methods and compositions of this disclosure can be used with any suitable digital processor. The digital processor can be programmed, for example, to operate any component of a device and/or execute methods described herein. In some cases, bead formation can be executed with the aid of a digital processor in communication with a droplet generator. The digital processor can control the speed at which droplets are formed or control the total number of droplets that are generated. In some cases, attaching barcode sequences to sample nucleic acid can be completed with the aid of a microfluidic device and a digital processor in communication with the microfluidic device. In some cases, the digital processor can control the amount of sample and/or beads provided to the channels of the microfluidic device, the flow rates of materials within the channels, and the rate at which droplets comprising barcode sequences and sample nucleic acid are generated.

The methods and compositions of this disclosure can be useful for a variety of different molecular biology applications including, but not limited to, nucleic acid sequencing, protein sequencing, nucleic acid quantification, sequencing optimization, detecting gene expression, quantifying gene expression, epigenetic applications, and single-cell analysis of genomic or expressed markers. Moreover, the methods and compositions of this disclosure can have numerous medical applications including identification, detection, diagnosis, treatment, staging of, or risk prediction of various genetic and non-genetic diseases and disorders including cancer.

### Emulsion Droplets

The methods, compositions and systems described herein may be useful for attaching barcodes, and particularly barcode nucleic acid sequences, to sample materials and/or components/fragments thereof. In general, this can be accomplished by partitioning sample material components/fragment into separate partitions or reaction volumes in which are co-partitioned a plurality of barcodes, which are then attached to sample components/fragment within the same partition. Methods to attach barcodes to sample components/fragments thereof may include ligation method, chain extension method, and transposase method.

In some cases, the partitions refer to containers or vessels (such as wells, microwells, tubes, vials, through ports in nanoarray substrates, e.g., BioTrove nanoarrays, or other containers). In some cases, the compartments or partitions comprise partitions that are flowable within fluid streams. These partitions may comprise, e.g., micro-vesicles that have an outer barrier surrounding an inner fluid center or core, or, in some cases, they may comprise a porous matrix that is capable of entraining and/or retaining materials within its matrix. In some aspects, partitions comprise droplets of aqueous fluid within a non-aqueous continuous phase, e.g., an oil phase. Examples of different vessels are described in U.S. Patent Application Publication No. 2014/0155295, which is entirely incorporated herein by reference. Examples of emulsion systems for creating stable droplets in non-aqueous or oil continuous phases are described in detail in U.S. Patent Application Publication No. 2010/0105112, which is entirely incorporated herein by reference.

In the case of droplets in an emulsion, allocating individual cells to discrete partitions may generally be accomplished by introducing a flowing stream of cells in an aqueous fluid into a flowing stream of a non-aqueous fluid, such that droplets are generated at the junction of the two streams. By providing the aqueous cell-containing stream at a certain concentration of cells, the occupancy of the resulting partitions (e.g., number of cells per partition) can be controlled. Where single cell partitions are desired, the relative flow rates of the fluids can be selected such that, on average, the partitions contain less than one cell per partition, in order to ensure that those partitions that are occupied, are primarily singly occupied. In some embodiments, the relative flow rates of the fluids can be selected such that a majority of partitions are occupied, e.g., allowing for only a small percentage of unoccupied partitions. In some aspects, the flows and channel architectures are controlled as to ensure a desired number of singly occupied partitions, less than a certain level of unoccupied partitions and less than a certain level of multiply occupied partitions.

The systems and methods described herein can be operated such that a majority of occupied partitions include no more than one cell per occupied partition. In some cases, the partitioning process is conducted such that fewer than 25% of the occupied partitions contain more than one cell, and in some cases, fewer than 20% of the occupied partitions have more than one cell. In some cases, fewer than 10% or fewer than 5% of the occupied partitions include more than one cell per partition.

In some cases, it is desirable to avoid the creation of excessive numbers of empty partitions. For example, from a cost perspective and/or efficiency perspective, it may desirable to minimize the number of empty partitions. While this may be accomplished by providing sufficient numbers of cells into the partitioning zone, the Poissonian distribution may expectedly increase the number of partitions that may include multiple cells. As such, in accordance with aspects described herein, the flow of one or more of the cells, or other fluids directed into the partitioning zone are conducted such that, in some cases, no more than 50% of the generated partitions, no more than 25% of the generated partitions, or no more than 10% of the generated partitions are unoccupied. Further, in some aspects, these flows are controlled so as to present non-Poissonian distribution of single occupied partitions while providing lower levels of unoccupied partitions. The above ranges of unoccupied partitions can be achieved while still providing any of the single occupancy rates described above. For example, the use of the systems and methods described herein creates resulting partitions that have multiple occupancy rates of less than or equal to about 25%, 20%, 15%, 10%, or 5%, while having unoccupied partitions of less than or equal to about 50%, 40%, 30%, 20%, 10%, or 5%.

As will be appreciated, the above-described occupancy rates are also applicable to partitions that include both cells and additional reagents and agents, including, but not limited to, microcapsules carrying barcoded oligonucleotides, microcapsules carrying anchoring oligonucleotides, labeling agents, labeling agents comprising reporter oligonucleotides, labeling agents comprising reporter oligonucleotides comprising a nucleic barcode sequence, and cells with one or more labeling agents bound to one or more cell surface features. In some aspects, a substantial percentage of the overall occupied partitions can include both a microcapsule (e.g., bead) comprising barcoded or anchoring oligonucleotides and a cell with or without bound labeling agents.

Although described in terms of providing substantially singly occupied partitions, above, in certain cases, it is desirable to provide multiply occupied partitions, e.g., containing two, three, four or more cells and/or microcapsules (e.g., beads) comprising barcoded oligonucleotides or anchor oligonucleotides within a single partition. Accordingly, the flow characteristics of the cell and/or bead containing fluids and partitioning fluids may be controlled to provide for such multiply occupied partitions. In particular, the flow parameters may be controlled to provide a desired occupancy rate at greater than or equal to about 50% of the partitions, greater than or equal to about 75%, or greater than or equal to about 80%, 90%, 95%, or higher.

In some cases, additional microcapsules are used to deliver additional reagents to a partition. In such cases, it may be advantageous to introduce different beads into a common channel or droplet generation junction, from different bead sources, i.e., containing different associated reagents, through different channel inlets into such common channel or droplet generation junction. In such cases, the flow and frequency of the different beads into the channel or junction may be controlled to provide for the desired ratio of microcapsules from each source, while ensuring the desired pairing or combination of such beads into a partition with the desired number of cells.

The partitions described herein may comprise small volumes, e.g., less than or equal to 10 μL, 5 μL, 1 μL, 900 picoliters (pL), 800 pL, 700 pL, 600 pL, 500 pL, 400pL, 300 pL, 200 pL, 100pL, 50 pL, 20 pL, 10 pL, 1 pL, 500 nanoliters (nL), 100 nL, 50 nL, or less.

For example, in the case of droplet based partitions, the droplets may have overall volumes that are less than or equal to 1000 pL, 900 pL, 800 pL, 700 pL, 600 pL, 500 pL, 400 pL, 300 pL, 200 pL, 100 pL, 50 pL, 20 pL, 10 pL, or 1 Pl. Where co-partitioned with microcapsules, it will be appreciated that the sample fluid volume, e.g., including co-partitioned cells, within the partitions may be less than or equal to 90%, 80%, 70%, 60%, 50%, 40%, 30%, 20%, 10%, or less than the above described volumes.

As is described elsewhere herein, partitioning species may generate a population or plurality of partitions. In such cases, any suitable number of partitions can be generated to generate the plurality of partitions. For example, in a method described herein, a plurality of partitions may be generated that comprises at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions or at least about 1,000,000,000 partitions. Moreover, the plurality of partitions may comprise both unoccupied partitions (e.g., empty partitions) and occupied partitions

Microfluidic channel networks, as described herein, may be utilized to generate partitions as described herein. Alternative mechanisms may also be employed in the partitioning of individual cells, including porous membranes through which aqueous mixtures of cells are extruded into non-aqueous fluids.

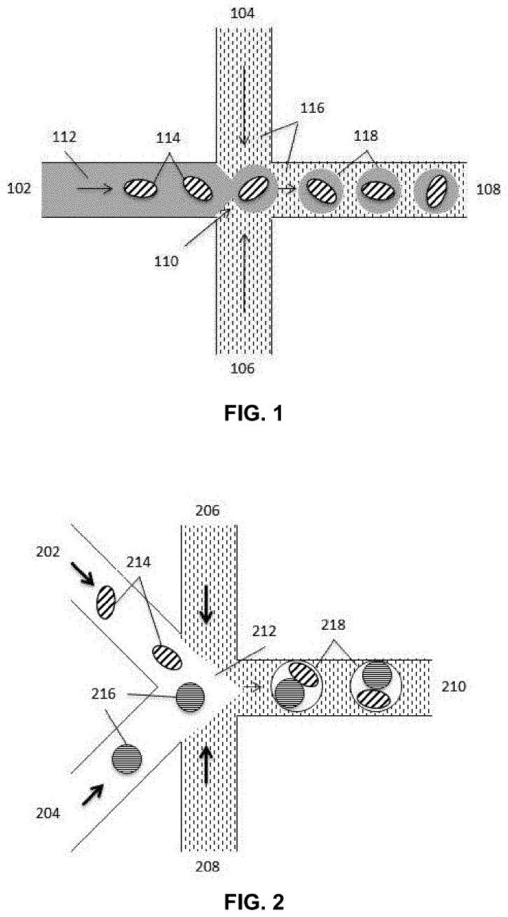

FIG. 1 illustrates an example of a simplified microfluidic channel structure for partitioning individual cells. Cells may be partitioned with or without labeling agents bound to cell surface features. As described herein, in some cases, the majority of occupied partitions include no more than one cell per occupied partition and, in some other cases, some of the generated partitions are unoccupied. In some cases, though, some of the occupied partitions may include more than one cell. In some cases, the partitioning process may be controlled such that fewer than 25% of the occupied partitions contain more than one cell, and in some cases, fewer than 20% of the occupied partitions have more than one cell, while in some cases, fewer than 10% or fewer than 5% of the occupied partitions include more than one cell per partition. As shown in FIG. 1, the channel structure can include channel segments 102, 104, 106 and 108 communicating at a channel junction 110. In operation, a first aqueous fluid 112 that includes suspended cells 114, may be transported along channel segment 102 into junction 110, while a second fluid 116 that is immiscible with the aqueous fluid 112 may be delivered to the junction 110 from channel segments 104 and 106 to create discrete droplets 118 of the aqueous fluid including individual cells 114, flowing into channel segment 108.

In some cases, this second fluid 116 may comprise an oil, such as a fluorinated oil, that includes a fluorosurfactant for stabilizing the resulting droplets, e.g., inhibiting subsequent coalescence of the resulting droplets. Examples of partitioning fluids and fluorosurfactants are described in U.S. Patent Application Publication No. 2010/0105112, which is entirely incorporated herein by reference.

In some cases, in addition to or as an alternative to droplet based partitioning, cells (with or without labeling agents bound to cell surface features, as described herein) may be encapsulated within a microcapsule that comprises an outer shell or layer or porous matrix in which is entrained one or more individual cells or small groups of cells, and may include other reagents. Encapsulation of cells may be carried out by a variety of processes. Such processes may combine an aqueous fluid containing the cells to be analyzed with a polymeric precursor material that may be capable of being formed into a gel or other solid or semi-solid matrix upon application of a particular stimulus to the polymer precursor. Such stimuli may include, e.g., thermal stimuli (either heating or cooling), photo-stimuli (e.g., through photo-curing), chemical stimuli (e.g., through crosslinking, polymerization initiation of the precursor (e.g., through added initiators), or the like.

Preparation of microcapsules comprising cells may be carried out by a variety of methods. For example, air knife droplet or aerosol generators may be used to dispense droplets of precursor fluids into gelling solutions in order to form microcapsules that include individual cells or small groups of cells. Likewise, membrane based encapsulation systems may be used to generate microcapsules comprising encapsulated cells as described herein. In some aspects, microfluidic systems like that shown in FIG. 1 may be readily used in encapsulating cells as described herein. In particular, and with reference to FIG. 1, the aqueous fluid comprising the cells and the polymer precursor materials may be flowed into channel junction 110, where it may be partitioned into droplets 118 comprising the individual cells 114, through the flow of non-aqueous fluid 116. In the case of encapsulation methods, non-aqueous fluid 116 may also include an initiator to cause polymerization and/or crosslinking of the polymer precursor to form the microcapsule that includes the entrained cells. Examples of polymer precursor/initiator pairs are described in U.S. Patent Application Publication No. 2014/0378345, which is entirely incorporated herein by reference.

For example, in the case where the polymer precursor material comprises a linear polymer material, e.g., a linear polyacrylamide, polyethylene glycol (PEG), or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams in channel segments 104 and 106, which initiates the copolymerization of the acrylamide and BAC into a cross-linked polymer network or, hydrogel.

Upon contact of the second fluid stream 116 with the first fluid stream 112 at junction 110 in the formation of droplets, the TEMED may diffuse from the second fluid 116 into the aqueous first fluid 112 comprising the linear polyacrylamide, which may activate the crosslinking of the polyacrylamide within the droplets, resulting in the formation of the gel, e.g., hydrogel, microcapsules 118, as solid or semi-solid beads or particles entraining the cells 114. Although described in terms of polyacrylamide encapsulation, other “activatable” encapsulation compositions may also be employed in the context of the methods and compositions described herein. For example, formation of alginate droplets followed by exposure to divalent metal ions, e.g., Ca²⁺, may be used as an encapsulation process using the described processes. Likewise, agarose droplets may also be transformed into capsules through temperature based gelling, e.g., upon cooling, or the like. In some cases, encapsulated cells may be selectively releasable from the microcapsule, e.g., through passage of time, or upon application of a particular stimulus, that degrades the microcapsule sufficiently to allow the cell, or its contents to be released from the microcapsule, e.g., into a partition, such as a droplet. For example, in the case of the polyacrylamide polymer described above, degradation of the microcapsule may be accomplished through the introduction of an appropriate reducing agent, such as DTT or the like, to cleave disulfide bonds that cross link the polymer matrix. See, e.g., U.S. Patent Application Publication No. 2014/0378345, which is entirely incorporated herein by reference.

Encapsulated cells or cell populations may provide certain advantages, such as being storable and more portable than droplet based partitioned cells. Furthermore, in some cases, it may be desirable to allow cells to be analyzed to incubate for a select period of time, in order to characterize changes in such cells over time, either in the presence or absence of different stimuli. In such cases, encapsulation of individual cells may allow for longer incubation than partitioning in emulsion droplets, although in some cases, droplet partitioned cells may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of cells may constitute the partitioning of the cells into which other reagents are co-partitioned. Alternatively, encapsulated cells may be readily deposited into other partitions, e.g., droplets, as described above.

In accordance with certain aspects, the cells may be partitioned along with lysis reagents in order to release the contents of the cells within the partition. In such cases, the lysis agents may be contacted with the cell suspension concurrently with, or immediately prior to the introduction of the cells into the partitioning junction/droplet generation zone, e.g., through an additional channel or channels upstream of channel junction 110. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the cells to cause the release of the cell's contents into the partitions. For example, in some cases, surfactant based lysis solutions may be used to lyse cells. These lysis surfactants may interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of cells that may be in addition to or in place of droplet partitioning, where any pore size of the encapsulate is sufficiently small to retain nucleic acid fragments of a desired size, following cellular disruption.

In addition to the lysis agents co-partitioned with the cells described above, other reagents may also be co-partitioned with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cells, the cells may be exposed to an appropriate stimulus to release the cells or their contents from a co-partitioned microcapsule. For example, in some cases, a chemical stimulus may be co-partitioned along with an encapsulated cell to allow for the degradation of the microcapsule and release of the cell or its contents into the larger partition. In some cases, this stimulus may be the same as the stimulus described elsewhere herein for release of oligonucleotides from their respective microcapsule (e.g., bead). In alternative aspects, this may be a different and non-overlapping stimulus, in order to allow an encapsulated cell to be released into a partition at a different time from the release of oligonucleotides into the same partition.

Additional reagents may also be co-partitioned with the cells, such as endonucleases to fragment the cell's DNA, DNA polymerase enzymes and dNTPs used to amplify the cell's nucleic acid fragments and to attach the barcode oligonucleotides to the amplified fragments. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In one example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

An example of a microfluidic channel structure for co-partitioning cells and beads comprising barcode oligonucleotides is schematically illustrated in FIG. 2. The channel structure may be part of a droplet generator. The droplet generator may be a chip, for example. The chip may be a consumable. In some cases, a subset of the overall occupied partitions may include both a bead and a cell and, in some cases, some of the partitions that are generated may be unoccupied. In some cases, some of the partitions may have beads and cells that are not partitioned 1:1. In some cases, it may be desirable to provide multiply occupied partitions, e.g., containing two, three, four or more cells and/or beads within a single partition. As shown, channel segments 202, 204, 206, 208 and 210 may be provided in fluid communication at channel junction (or intersection) 212. An aqueous stream comprising the individual cells 214 may flow through channel segment 202 toward channel junction 212. As described above, these cells may be suspended within an aqueous fluid, or may have been pre-encapsulated, prior to the partitioning process.

Concurrently, an aqueous stream comprising the barcode carrying beads 216, may flow through channel segment 204 toward channel junction 212. A non-aqueous partitioning fluid 216 may be introduced into channel junction 212 from each of side channels 206 and 208, and the combined streams may flow into outlet channel 210. Within channel junction 212, the two combined aqueous streams from channel segments 202 and 204 may be combined, and partitioned into droplets/partitions 218, that may include co-partitioned cells 214 and beads 216. By controlling the flow characteristics of each of the fluids combining at channel junction 212, as well as controlling the geometry of the channel junction, partitioning can be optimized to achieve a desired occupancy level of beads, cells or both, within the droplets/partitions 218 that are generated.

In some cases, lysis agents, e.g., cell lysis enzymes, may be introduced into the partition with the bead stream, e.g., flowing through channel segment 204, such that the cell may be lysed at or after the time of partitioning. In some cases, cell membranes may be maintained intact, such as to allow for the characterization of cell surface markers. Additional reagents may also be added to the partition in this configuration, such as endonucleases to fragment the cell's DNA, DNA polymerase enzyme and dNTPs used to amplify the cell's nucleic acid fragments and to attach the barcode oligonucleotides to the amplified fragments. A chemical stimulus, such as DTT, may be used to release the barcodes from their respective beads into the partition. In such cases, it may be particularly desirable to provide the chemical stimulus along with the cell-containing stream in channel segment 202, such that release of the barcodes only occurs after the two streams have been combined, e.g., within the droplets/partitions 218. Where the cells are encapsulated, however, introduction of a common chemical stimulus, e.g., that both releases the oligonucleotides form their beads, and releases cells from their microcapsules may generally be provided from a separate additional side channel (not shown) upstream of or connected to channel junction 212.

A number of other reagents may be co-partitioned along with the cells, beads, lysis agents and chemical stimuli, including, for example, protective reagents, like proteinase K, chelators, nucleic acid extension, replication, transcription or amplification reagents such as polymerases, reverse transcriptases, transposases which can be used for transposon based methods (e.g., Nextera), nucleoside triphosphates or NTP analogues, primer sequences and additional cofactors such as divalent metal ions used in such reactions, ligation reaction reagents, such as ligase enzymes and ligation sequences, dyes, labels, or other tagging reagents.

The channel networks, e.g., as described herein, can be fluidly coupled to appropriate fluidic components. For example, the inlet channel segments, e.g., channel segments 202, 204, 206 and 208 may be fluidly coupled to appropriate sources of the materials they are to deliver to channel junction 212. For example, channel segment 202 may be fluidly coupled to a source of an aqueous suspension of cells 214 to be analyzed, while channel segment 204 may be fluidly coupled to a source of an aqueous suspension of beads 216. Channel segments 206 and 208 may then be fluidly connected to one or more sources of the non-aqueous fluid. These sources may include any of a variety of different fluidic components, from simple reservoirs defined in or connected to a body structure of a microfluidic device, to fluid conduits that deliver fluids from off-device sources, manifolds, or the like. Likewise, the outlet channel segment 210 may be fluidly coupled to a receiving vessel or conduit for the partitioned cells. Again, this may be a reservoir defined in the body of a microfluidic device, or it may be a fluidic conduit for delivering the partitioned cells to a subsequent process operation, instrument or component.

As an alternative, the channel segments 202 and 204 may meet at another junction upstream of the junction 212. At such junction, beads and biological particles may form a mixture that is directed along another channel to the junction 212 to yield droplets/partitions 218. The mixture may provide the beads and biological particles in an alternating fashion, such that, for example, a droplet comprises a single bead and a single biological particle.

As an alternative to the droplet generators of FIGS. 1 and 2, an emulsion may be generated by flowing a first fluid phase along a first channel towards an intersection of the first channel with a second channel or a collection chamber (or vessel). The second channel or collection chamber may include a second fluid phase that is immiscible with the first fluid phase. At the intersection, the first fluid phase may come in contact with the second fluid phase to yield the emulsion comprising a plurality of droplets. The plurality of droplets may be pooled or collected in the second channel or collection chamber, or subjected to flow in the second channel along a direction leading away from the intersection.

A plurality of droplets may be subjected to flow or directed along a channel with the aid of a fluid flow system. Such fluid flow system may include one or more pumps for supplying negative pressure, one or more compressors for supplying positive pressure, or a combination of both. In some cases, A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

In an example process, a first partition can be provided that can include a plurality of first oligonucleotides (e.g., nucleic acid barcode molecules) that each can comprise a common nucleic acid barcode sequence. The first partition can comprise any of a variety of portable partitions, e.g., a bead (e.g., a degradable bead, a gel bead), a droplet (e.g., an aqueous droplet in an emulsion), a microcapsule, or the like, to which the first oligonucleotides are releasably attached, releasably coupled, or are releasably associated. Moreover, any suitable number of first oligonucleotides can be included in the first partition. For example, the first oligonucleotides can be releasably attached to, releasably coupled to, or releasably associated with the first partition via a cleavable linkage such as, for example, a chemically cleavable linkage (e.g., a disulfide linkage, or any other type of chemically cleavable linkage), a photocleavable linkage, and/or a thermally cleavable linkage. In some cases, the first partition can be a bead and the bead can be a degradable bead (e.g., a photodegradable bead, a chemically degradable bead, a thermally degradable bead, or any other type of degradable bead). Moreover, the bead can comprise chemically-cleavable cross-linking (e.g., disulfide cross-linking).

The first partition can then be co-partitioned into a second partition, together with a sample material, sample material component, fragment of a sample material, or a fragment of a sample material component. The sample material (or component or fragment thereof) can be any appropriate sample type. In cases where a sample material or component of a sample material comprises one or more nucleic acid fragments, the one or more nucleic acid fragments can be of any suitable length. The second partition can include any of a variety of partitions, including for example, wells, microwells, nanowells, tubes or containers, or in some cases droplets (e.g., aqueous droplets in an emulsion) or microcapsules in which the first partition can be co-partitioned. In some cases, the first partition can be provided in a first aqueous fluid and the sample material, sample material component, or fragment of a sample material component can be provided in a second aqueous fluid. During co-partitioning, the first aqueous fluid and second aqueous fluid can be combined within a droplet within an immiscible fluid. In some cases, the second partition can comprise no more than one first partition. In some cases, the second partition can comprise no more than one, two, three, four, five, six, seven, eight, nine, or ten first partitions. In some cases, the second partition can comprise at least one, two, three, four, five, six, seven, eight, nine, ten, or more first partitions.

Once co-partitioned, the first oligonucleotides comprising the barcode sequences can be released from the first partition (e.g., via degradation of the first partition, cleaving a chemical linkage between the first oligonucleotides and the first partition, or any other suitable type of release) into the second partition, and attached to the sample components co-partitioned therewith. In some cases, the first partition can comprise a bead and the crosslinking of the bead can comprise a disulfide linkage. In addition, or as an alternative, the first oligonucleotides can be linked to the bead via a disulfide linkage. In either case, the first oligonucleotides can be released from the first partition by exposing the first partition to a reducing agent (e.g., dithiothreitol (DTT) or tris(2-carboxyethyl)phosphine (TCEP)).

Attachment of the barcodes to sample components can include the direct attachment of the barcode oligonucleotides to sample materials, e.g., through ligation, hybridization, or other associations. Additionally, in many cases, for example, in barcoding of nucleic acid sample materials (e.g., template nucleic acid sequences, template nucleic acid molecules), components or fragments thereof, such attachment can additionally comprise the use of the barcode-containing oligonucleotides as priming sequences. The priming sequence can be complementary to at least a portion of a nucleic acid sample material and can be extended along the nucleic acid sample materials to create complements to such sample materials, as well as at least partial amplification products of those sequences or their complements.

In another example process, a plurality of first partitions can be provided that comprise a plurality of different nucleic acid barcode sequences. Each of the first partitions can comprise a plurality of nucleic acid barcode molecules having the same nucleic acid barcode sequence associated therewith. Any suitable number of nucleic acid barcode molecules can be associated with each of the first partitions, including, for example, at least about 2, 10, 100, 500, 1000, 5000, 10000, 50000, 100000, 500000, 1000000, 5000000, 10000000, 50000000, or 1000000000, or more than 1000000000 different nucleic acid barcode sequences.

As discussed above, the first partitions can be co-partitioned with sample materials, fragments of a sample material, components of a sample material, or fragments of a component(s) of a sample material into a plurality of second partitions. In some cases, a subset of the second partitions can comprise the same nucleic acid barcode sequence. For example, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more than 95% of the second partitions can comprise the same nucleic acid barcode sequence. Moreover, the distribution of first partitions per second partition can also vary according to, for example, occupancy rates described elsewhere herein. In cases where the plurality of first partitions comprises a plurality of different first partitions, each different first partition can be disposed within a separate second partition.

Following co-partitioning, the nucleic acid barcode molecules associated with the first partitions can be released into the plurality of second partitions. The released nucleic acid barcode molecules can then be attached to the sample materials, sample material components, fragments of a sample material, or fragments of sample material components, within the second partitions. In the case of barcoded nucleic acid species (e.g., barcoded sample nucleic acid, barcoded template nucleic acid, barcoded fragments of one or more template nucleic acid sequences, etc.), the barcoded nucleic acid species can be sequenced.

In another example process, an activatable nucleic acid barcode sequence can be provided and partitioned with one or more sample materials, components of a sample material, fragments of a sample material, or fragments of a component(s) of a sample material into a first partition. With the first partition, the activatable nucleic acid barcode sequence can be activated to produce an active nucleic acid barcode sequence. The active nucleic acid barcode sequence can then be attached to the one or more sample materials, components of a sample material, fragments of a sample material, or fragments of a component(s) of a sample material.

In some cases, the activatable nucleic acid barcode sequence can be coupled to a second partition that is also partitioned in the first partition with the activatable nucleic acid barcode sequence. An activatable nucleic acid barcode sequence can be activated by releasing the activatable nucleic acid barcode sequence from an associated partition (e.g., a bead). Thus, in cases where an activatable nucleic acid barcode sequence is associated with a second partition (e.g., a bead) that is partitioned in a first partition (e.g., a fluidic droplet), the activatable nucleic acid barcode sequence can be activated by releasing the activatable nucleic acid barcode sequence from its associated second partition. In addition, or as an alternative, an activatable barcode can also be activated by removing a removable blocking or protecting group from the activatable nucleic acid barcode sequence.

### Process of Barcoding Fragments of Template Nucleic Acids

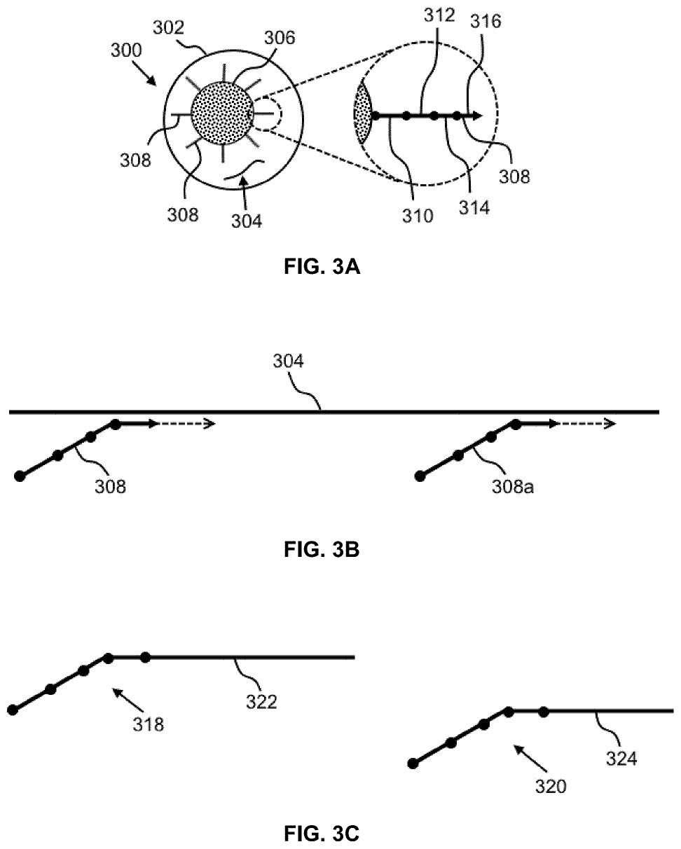

The present disclosure provides methods, and systems for preparing sequencing samples from sample nucleic acids using stabilized emulsion droplets. In some cases, as shown in FIGS. 3A-3C and as described in U.S. patent application Ser. No. 14/316,383, filed Jun. 26, 2014, which is entirely incorporated herein by reference, an example process to prepare barcoded fragments of template nucleic acids as a set of sequencing samples is shown using droplets 300. As shown in FIG. 3A, the droplet 300 may comprise a barrier layer 302 enclosing a sample nucleic acid 304 co-partitioned with a bead 306 in a droplet 300 in an emulsion. Within the droplet 300, oligonucleotides 308 may be provided on the bead 306. The oligonucleotides 308 may be released from the bead 306 and become reagents within the droplet 300. As shown in FIG. 3A, each oligonucleotide 308 may include a barcode sequence 332, in addition to one or more functional sequences, e.g., sequences 330, 334 and 336. For example, sequence 330 may function as an attachment or immobilization sequence for a given sequencing system, e.g., a P5 sequence used for attachment in flow cells of an Illumina Hiseq or Miseq system. Sequence 336 may be a primer such as, for example, a universal, random or targeted N-mer for priming replication of portions of the sample nucleic acid 304. Sequence 334 may provide a sequencing priming region, such as a “read1” or R1 priming region that is used to prime polymerase mediated, template directed sequencing by synthesis reactions in sequencing systems. In many cases, the barcode sequence 312, immobilization sequence 310 and R1 sequence 314 may be common to all of the oligonucleotides 308 attached to a given bead. The primer sequence 316 may vary for random N-mer primers, or may be common to the oligonucleotides 308 on a given bead for certain targeted applications. Although described with reference to the specific positioning and type of functional sequence segment elements within the barcode oligonucleotides 308, the position and nature of the functional segments within a barcode oligonucleotide 308 may vary. For example, primer sequences for different sequencing systems may be employed in place of the P5 or read1 primers. Additionally, in some cases, the positional context of the different segments may be changed. For example, in some cases, the barcode sequence segment 312 may be placed at the 5′ end of the sequence read primer or R1 segment 314, e.g., between segments 314 and 316, so that the barcode can be sequenced in a first pass or initial sequence read, e.g., following priming of the read1 sequence during the sequencing of the resultant barcoded fragments, as opposed to obtaining the barcode read on a subsequent sequencing read of a reverse complement.

Based upon the presence of primer sequence 316, the oligonucleotides 308 and 308a may be able to prime the sample nucleic acid 304 as shown in FIG. 3B, which may allow for extension of the oligonucleotides 308 and 308a annealed on the sample nucleic acid 304 in the presence of polymerase enzymes and other extension reagents, which may also be co-partitioned with the bead 306 and sample nucleic acid 304. The polymerase enzymes may include thermostable polymerases, e.g., where initial denaturation of double stranded sample nucleic acids within the partitions is desired. Alternatively, denaturation of sample nucleic acids may precede partitioning, such that single stranded target nucleic acids may be deposited into the partitions, allowing the use of non-thermostable polymerase enzymes, e.g., Klenow, phi29 DNA polymerase, DNA polymerase lambda (Poll), and the like. As shown in FIG. 3B, extension of the oligonucleotides 308 and 308a may anneal to multiple different regions of the sample nucleic acid 304. Consequently, multiple overlapping complements or fragments of the sample nucleic acid 304 can be created, e.g., fragments 318 and 320 as shown in FIG. 3C. Although fragments 318 and 320 may comprise sequences that are complementary to sample nucleic acid 304, e.g., insert sequences 322 and 324 (also referred to as “inserts”), these fragments herein may generally be referred to as comprising fragments of the sample nucleic acid 304, having the attached barcode sequences. These insert sequences 322 and 324 may then be subjected to sequence analysis, or they may be subjected to further processing.

### Surface-Mediated Coalescence of Emulsion Droplets

The emulsions of the present disclosure may include discontinuous aqueous and/or lipophilic (e.g., hydrocarbon) droplets in a continuous, fluorophilic phase. In other words, separate, isolated regions of droplets of an aqueous and/or lipophilic component are contained within a continuous fluorophilic phase, which may be defined by a fluorocarbon component. The discontinuous aqueous and/or lipophilic droplets in the nonaqueous phase may have an average cross-sectional dimension of greater than 25 nm. In some cases, the average cross-sectional dimension of the droplets may be greater than 50 nm, greater than 100 nm, greater than 250 nm, greater than 500 nm, greater than 1 micron, greater than 5 microns, greater than 10 microns, greater than 50 microns, greater than 100 microns, greater than 200 microns, or greater than 500 microns, etc. As used herein, the average cross-sectional dimension of a droplet generally refers to the diameter of a perfect sphere having the same volume as the droplet.

Emulsion droplets of the present disclosure may be stable for at least about 1 minute, at least about 5 minutes, at least about 10 minutes, at least about 20 minutes, at least about 30 minutes, at least about 40 minutes, at least about 1 hour, at least about 2 hours, at least about 6 hours, at least about 12 hours, at least about 1 day, at least about 1 week, at least about 1 month, or at least about 2 months, at a temperature of about 25 degrees Celsius and a pressure of 1 atm. As used herein, the term “stable emulsion” generally refers to emulsion compositions of which at least about 95% of the droplets do not coalesce, e.g., to form larger droplets over these periods of time when compared with the average size of droplets.

As described above, for long-term compartmentalization of reagents/components in droplets, surfactants may be added to reduce coalescence of droplets in a multi-phase (e.g., two-phase) emulsion system. Such multi-phase emulsion system may include a plurality of fluid phases, such as a first fluid phase and a second fluid phase that is immiscible. The role of the surfactant molecules in stabilizing emulsion droplets may be to increase the height of the energy barrier between the local energy minima of the system and its global minimum; this minimum may be reached with a system where both phases are separated by an interface of minimal energy and in which the chemical potentials of all species is homogeneous. P. Gruner, et al., “Controlling molecular transport in minimal emulsions;” Nat. Commun. (2016) 7:10392. Factors driving the emulsions toward equilibrium may include flocculation, coalescence, gravitational separation, Ostwald ripening, and solute transport. The kinetic stabilization of emulsions may occur through several mechanisms, including, for example, electrostatic or steric repulsion and the buildup of Marangoni stresses to improve the lifetime of emulsions against coalescence.

Despite the surfactant-induced stability, many factors may cause coalescence of droplet pairs in close vicinity. For example, perfluorobutanol has been reported to cause bulk coalescence with no mixing or centrifugation. See I. Akartuna, et al, “Chemically induced coalescence in droplet-based microfluidics,” Lab Chip (2015) 15:1140-114.

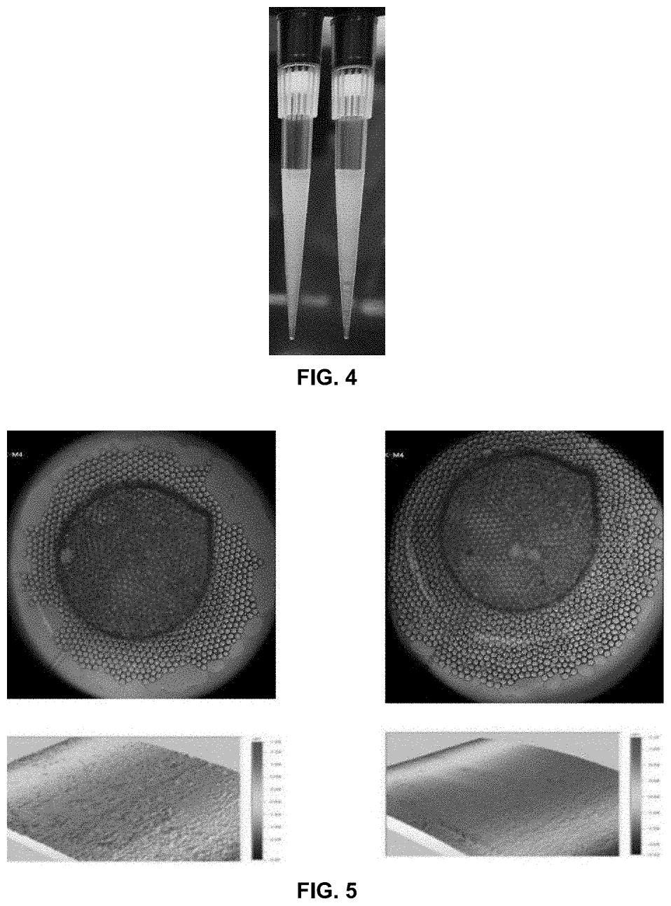

FIG. 4 shows various examples of emulsion formation with observed coalescence of droplets. The emulsions are formed using fluorinated oil and aqueous solution in a single-cell-based nucleic acid analysis platform. This figures shows emulsion droplets near the bottom of the pipet tip on the right coalescing, i.e., to form larger droplets.

The observed coalescence may be surface-mediated. For example, as shown in FIG. 5, when the surface roughness increases, so does the extent of droplet coalescence. The top left panel of FIG. 5 shows an image of an emulsion over surface A of a well/chip under the microscope. The bottom left panel of FIG. 5 depicts an image of the surface roughness of surface A. The top right panel and the bottom right panel show an image of an emulsion over surface B of another well/chip under the microscope and an image of the surface roughness of surface B, respectively. Observing the top two panels indicates that there are many large droplets at the peripheral/edge of the well/chip where the droplets are in contact with the well/chip surface. In addition, the extent of coalescence in the image of the top-left panel is more than that in the image of the top-right panel. Further, comparison of the bottom panels shows that surface A of the left panel has greater roughness than surface B of the right panel. FIG. 5 shows that the observed coalescence of emulsion droplets in emulsion wells may be surface-mediated coalescence.

As used herein, the term “surface-mediated coalescence” generally refers to the merger of two or more droplets/Gel bead-in-droplets due to their contact with a roughened surface to form a single daughter droplet. Such coalescence may result in loss of partitioning behavior for those affected particular droplets/Gel bead-in-droplets and a failed assay when the coalescence is not intended. In other cases, such coalescence may be necessary to recombine reaction components in two or more droplets before proceeding with further sample processing, making surface-mediated coalescence a useful outcome under the circumstance. Nevertheless, discovering factors that may control the coalescence behavior of an emulsion system may be desirable in either situation.

A formulation of the emulsion may comprise an oil phase and an aqueous phase. To decrease the observed coalescence of droplets, various alternative reagents/components in the aqueous phase may be changed. For example, different grades and concentrations of different surfactants such as SYNPERONIC® surfactants may be used. Kinetically sacrificial molecules such as glycoside may be added. Different enzymes, such as, for example, bovine serum albumin (BSA) and/or lysozyme, may be included to populate the droplet boundary. Different lysis agents, such as, for example, glycosides and maltosides with alkyl chains, may also be used. Although reducing coalescence by varying components of the aqueous phase is desirable, other intended reactions within or associated with the droplets may be considered simultaneously.

As used herein the terms “critical micelle concentration” or CMC generally refers to the minimal concentration, above which a surfactant can form micelles at a specific temperature. Different lysis agents may have different CMC's. Different lysis agents may also behave differently when lysing cells at or above their respective CMC's. For example, n-dodecyl-β-D-maltoside (DBDM) has a CMC of about 0.17 mM. DBDM is also an effective lysis agent at a formulation concentration of about 0.5% w/v. Detergents with structures similar to DBDM may or may not achieve cell lysis in emulsion droplets at formulation concentrations of about 0.5% w/v. In addition, detergents with structures similar to DBDM may or may not stabilize emulsion droplets to the same extent as DBDM does. The presence of DBDM may be associated with the observed surface-mediated coalescence. Other lysis agents, when used at formulation concentrations of about 0.5% w/v, do not lead to coalescence to the same extent of when DBDM is used.

The structure of n-dodecyl-β-D-maltoside (DBDM) is:

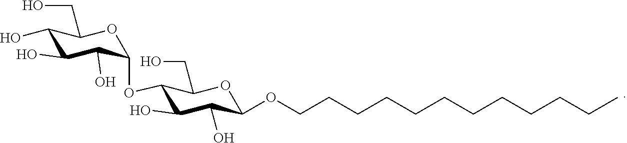

Detergent-based cell lysis may be a milder and easier alternative to physical disruption of cell membranes. It may be used in conjunction with homogenization and mechanical grinding. Detergents may break the lipid barrier surrounding cells by solubilizing proteins and disrupting lipid:lipid, protein:protein and protein:lipid interactions. Detergents, like lipids, may self-associate and bind to hydrophobic surfaces. They may comprise a polar hydrophilic head group and a nonpolar hydrophobic tail. As a result, they may be categorized by the nature of the head group as either ionic (cationic or anionic), nonionic or zwitterionic. Lysis agents may include, but not be limited to, hemifluorinated maltosides, zwitterionic agents (comprising both negative and positive charges), and tripod amphiphile. See, P. D. Laible, “Tripod Amphiphiles for Membrane Protein Manipulation,” Mol. Biosyst. (2010) 6:89-94.

Various components of the oil phase can be changed as well. The oil phase may comprise a fluorinated base oil, such as, for example, 3-ethoxyperfluoro(2-methylhexane) or HFE-7500 engineered oil, and a fluorinated surfactant, a tri-block surfactant used to stabilize droplets/Gel beads-in-emulsions (GEM's). Alternative choices for these oil phase components may include, for example, fluorous-phase soluble nano-particles that may provide steric bulk and prevent drops/GEM's from contacting well/chip surface; sacrificial co-surfactant that may cover the well/chip surface to reduce contact between droplets/GEM's with the well/chip surface, hydrophobic additives, different concentrations for the surfactant, different surfactants, and different fluorinated oils. Although reducing coalescence by varying components of the oil phase is desirable, other intended reactions within or associated with the droplets may be considered simultaneously.

For example, fluorous-phase soluble silica nano-particles may be soluble in the fluorinated base oil used. Nano-particles may be functionalized with triethoxy(1H,1H,2H,2H-perfluorooctyl)silane in the presence of a base in ethanol to put multiple copies of the (1H,1H,2H,2H-perfluorooctyl)silane moieties on the surface of about 200 nm mesoporous silica nano-particles. Nano-particles thus functionalized may give the following example partial structure wherein the circle represents the nano-particle:

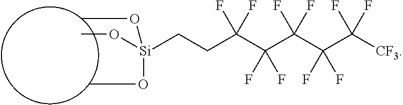

Sacrificial co-surfactant may be a mixture of fluorinated carboxylic acids of the following structure:

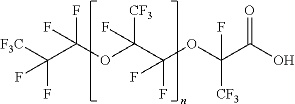

wherein n is an integer from 8 to 18. In some cases, the average of n may be about 12.2. These sacrificial co-surfactants may be kinetically favorable to migrate to the surface of the well containing the emulsion, partially due to its smaller average molecular weight of about 2350 g/mol when compared with the average molecular weight of the tri-block fluorinated surfactant used at about 6420 g/mol.

Hydrophobic additives may include 1-(perfluorodecyl)octane and its analogues, and compounds of the following structure:

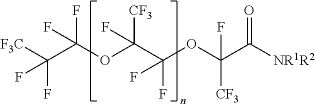

wherein n is an integer from 8 to 42, R¹ is H or octyl, and R² is octyl, decyl, dodecyl, or octadecyl. In some cases, the average of n may be about 12. In other cases, the average of n may be about 36. In some cases, both R¹ and R² may be octyl. In some cases, the hydrophobic additives may be added to the partitioning oil for final concentrations of about 0.01 mM, about 0.1 mM or about 1.0 mM.

### Tri-Block Fluorinated Surfactants

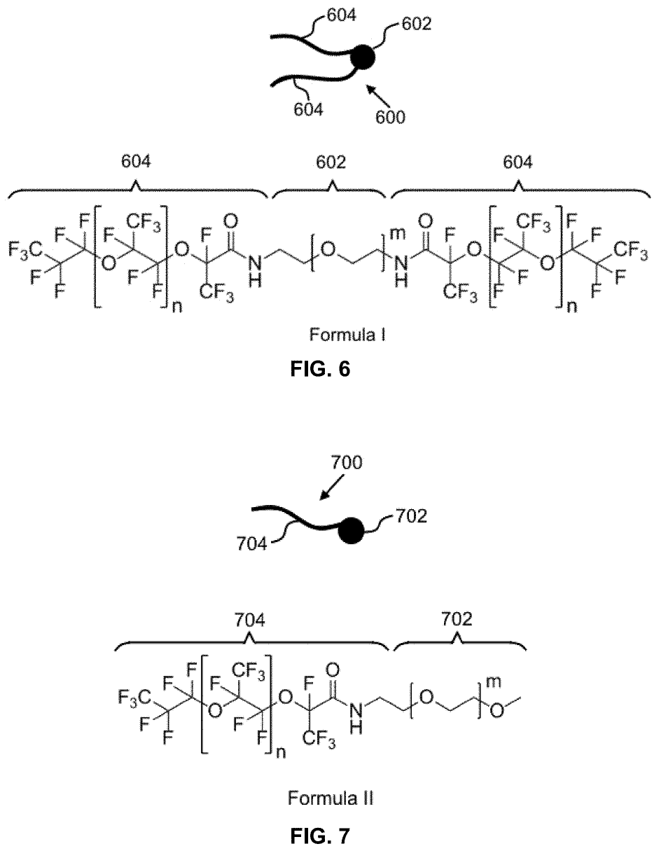

FIG. 6 shows an example of fluorinated surfactant 600 used in the partitioning oil phase. The fluorinated surfactant 600 is a tri-block surfactant comprising a hydrophilic head group 602 and two fluorophilic tails 604. An example of the fluorinated surfactant 600 is shown in FIG. 6 as Formula I:

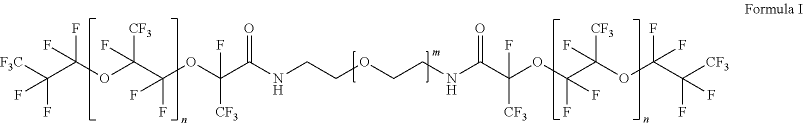

wherein m is an integer from 5 to 50 and n is an integer from 5 to 60. In Formula I, two fluorophilic tails of perfluoropolyether (PFPE) chains 604 are linked through amide bonds to a hydrophilic head group of polyethylene glycol (PEG) group 602 to form the tri-block surfactant 600.

### Di-Block Fluorinated Surfactants

FIG. 7 shows another type of surfactant, fluorinated surfactant 700, which is a di-block surfactant (or di-block copolymer) comprising a hydrophilic head group 702 and only one fluorophilic tail 704. An example of the fluorinated surfactant 700 is shown in FIG. 7 as Formula II:

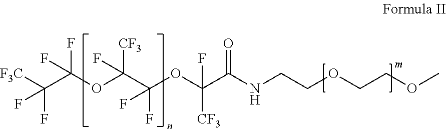

wherein m is an integer from 5 to 50; and n is an integer from 5 to 60. It should be noted that the fluorinated surfactant 700, such as, for example, compounds of Formula II, may be a mixture of compounds with varying values for integers m and n. In some cases, the fluorinated surfactant 700, such as, for example, compounds of Formula II, wherein m is an integer from 10 to 22 and n is an integer from 30 to 42, m is an integer from 12 to 20 and n is an integer from 32 to 40, m is an integer from 14 to 18 and n is an integer from 34 to 38, m is 15, 16, 17 and n is 35, 36, 37. In some cases, values for integers (m/n) for compounds of Formula II can be (5/5), (5/6), (5/7), (5/8), (5/9), (5/10), (5/11), (5/12), (5/13), (5/14), (5/15), (5/16), (5/17), (5/18), (5/19), (5/20), (5/21), (5/22), (5/23), (5/24), (5/25), (5/26), (5/27), (5/28), (5/29), (5/30), (5/31), (5/32), (5/33), (5/34), (5/35), (5/36), (5/37), (5/38), (5/39), (5/40), (5/41), (5/42), (5/43), (5/44), (5/45), (5/46), (5/47), (5/48), (5/49), (5/50), (5/51), (5/52), (5/53), (5/54), (5/55), (5/56), (5/57), (5/58), (5/59), (5/60), (6/5), (6/6), (6/7), (6/8), (6/9), (6/10), (6/11), (6/12), (6/13), (6/14), (6/15), (6/16), (6/17), (6/18), (6/19), (6/20), (6/21), (6/22), (6/23), (6/24), (6/25), (6/26), (6/27), (6/28), (6/29), (6/30), (6/31), (6/32), (6/33), (6/34), (6/35), (6/36), (6/37), (6/38), (6/39), (6/40), (6/41), (6/42), (6/43), (6/44), (6/45), (6/46), (6/47), (6/48), (6/49), (6/50), (6/51), (6/52), (6/53), (6/54), (6/55), (6/56), (6/57), (6/58), (6/59), (6/60), (7/5), (7/6), (7/7), (7/8), (7/9), (7/10), (7/11), (7/12), (7/13), (7/14), (7/15), (7/16), (7/17), (7/18), (7/19), (7/20), (7/21), (7/22), (7/23), (7/24), (7/25), (7/26), (7/27), (7/28), (7/29), (7/30), (7/31), (7/32), (7/33), (7/34), (7/35), (7/36), (7/37), (7/38), (7/39), (7/40), (7/41), (7/42), (7/43), (7/44), (7/45), (7/46), (7/47), (7/48), (7/49), (7/50), (7/51), (7/52), (7/53), (7/54), (7/55), (7/56), (7/57), (7/58), (7/59), (7/60), (8/5), (8/6), (8/7), (8/8), (8/9), (8/10), (8/11), (8/12), (8/13), (8/14), (8/15), (8/16), (8/17), (8/18), (8/19), (8/20), (8/21), (8/22), (8/23), (8/24), (8/25), (8/26), (8/27), (8/28), (8/29), (8/30), (8/31), (8/32), (8/33), (8/34), (8/35), (8/36), (8/37), (8/38), (8/39), (8/40), (8/41), (8/42), (8/43), (8/44), (8/45), (8/46), (8/47), (8/48), (8/49), (8/50), (8/51), (8/52), (8/53), (8/54), (8/55), (8/56), (8/57), (8/58), (8/59), (8/60), (9/5), (9/6), (9/7), (9/8), (9/9), (9/10), (9/11), (9/12), (9/13), (9/14), (9/15), (9/16), (9/17), (9/18), (9/19), (9/20), (9/21), (9/22), (9/23), (9/24), (9/25), (9/26), (9/27), (9/28), (9/29), (9/30), (9/31), (9/32), (9/33), (9/34), (9/35), (9/36), (9/37), (9/38), (9/39), (9/40), (9/41), (9/42), (9/43), (9/44), (9/45), (9/46), (9/47), (9/48), (9/49), (9/50), (9/51), (9/52), (9/53), (9/54), (9/55), (9/56), (9/57), (9/58), (9/59), (9/60), (10/5), (10/6), (10/7), (10/8), (10/9), (10/10), (10/11), (10/12), (10/13), (10/14), (10/15), (10/16), (10/17), (10/18), (10/19), (10/20), (10/21), (10/22), (10/23), (10/24), (10/25), (10/26), (10/27), (10/28), (10/29), (10/30), (10/31), (10/32), (10/33), (10/34), (10/35), (10/36), (10/37), (10/38), (10/39), (10/40), (10/41), (10/42), (10/43), (10/44), (10/45), (10/46), (10/47), (10/48), (10/49), (10/50), (10/51), (10/52), (10/53), (10/54), (10/55), (10/56), (10/57), (10/58), (10/59), (10/60), (11/5), (11/6), (11/7), (11/8), (11/9), (11/10), (11/11), (11/12), (11/13), (11/14), (11/15), (11/16), (11/17), (11/18), (11/19), (11/20), (11/21), (11/22), (11/23), (11/24), (11/25), (11/26), (11/27), (11/28), (11/29), (11/30), (11/31), (11/32), (11/33), (11/34), (11/35), (11/36), (11/37), (11/38), (11/39), (11/40), (11/41), (11/42), (11/43), (11/44), (11/45), (11/46), (11/47), (11/48), (11/49), (11/50), (11/51), (11/52), (11/53), (11/54), (11/55), (11/56), (11/57), (11/58), (11/59), (11/60), (12/5), (12/6), (12/7), (12/8), (12/9), (12/10), (12/11), (12/12), (12/13), (12/14), (12/15), (12/16), (12/17), (12/18), (12/19), (12/20), (12/21), (12/22), (12/23), (12/24), (12/25), (12/26), (12/27), (12/28), (12/29), (12/30), (12/31), (12/32), (12/33), (12/34), (12/35), (12/36), (12/37), (12/38), (12/39), (12/40), (12/41), (12/42), (12/43), (12/44), (12/45), (12/46), (12/47), (12/48), (12/49), (12/50), (12/51), (12/52), (12/53), (12/54), (12/55), (12/56), (12/57), (12/58), (12/59), (12/60), (13/5), (13/6), (13/7), (13/8), (13/9), (13/10), (13/11), (13/12), (13/13), (13/14), (13/15), (13/16), (13/17), (13/18), (13/19), (13/20), (13/21), (13/22), (13/23), (13/24), (13/25), (13/26), (13/27), (13/28), (13/29), (13/30), (13/31), (13/32), (13/33), (13/34), (13/35), (13/36), (13/37), (13/38), (13/39), (13/40), (13/41), (13/42), (13/43), (13/44), (13/45), (13/46), (13/47), (13/48), (13/49), (13/50), (13/51), (13/52), (13/53), (13/54), (13/55), (13/56), (13/57), (13/58), (13/59), (13/60), (14/5), (14/6), (14/7), (14/8), (14/9), (14/10), (14/11), (14/12), (14/13), (14/14), (14/15), (14/16), (14/17), (14/18), (14/19), (14/20), (14/21), (14/22), (14/23), (14/24), (14/25), (14/26), (14/27), (14/28), (14/29), (14/30), (14/31), (14/32), (14/33), (14/34), (14/35), (14/36), (14/37), (14/38), (14/39), (14/40), (14/41), (14/42), (14/43), (14/44), (14/45), (14/46), (14/47), (14/48), (14/49), (14/50), (14/51), (14/52), (14/53), (14/54), (14/55), (14/56), (14/57), (14/58), (14/59), (14/60), (15/5), (15/6), (15/7), (15/8), (15/9), (15/10), (15/11), (15/12), (15/13), (15/14), (15/15), (15/16), (15/17), (15/18), (15/19), (15/20), (15/21), (15/22), (15/23), (15/24), (15/25), (15/26), (15/27), (15/28), (15/29), (15/30), (15/31), (15/32), (15/33), (15/34), (15/35), (15/36), (15/37), (15/38), (15/39), (15/40), (15/41), (15/42), (15/43), (15/44), (15/45), (15/46), (15/47), (15/48), (15/49), (15/50), (15/51), (15/52), (15/53), (15/54), (15/55), (15/56), (15/57), (15/58), (15/59), (15/60), (16/5), (16/6), (16/7), (16/8), (16/9), (16/10), (16/11), (16/12), (16/13), (16/14), (16/15), (16/16), (16/17), (16/18), (16/19), (16/20), (16/21), (16/22), (16/23), (16/24), (16/25), (16/26), (16/27), (16/28), (16/29), (16/30), (16/31), (16/32), (16/33), (16/34), (16/35), (16/36), (16/37), (16/38), (16/39), (16/40), (16/41), (16/42), (16/43), (16/44), (16/45), (16/46), (16/47), (16/48), (16/49), (16/50), (16/51), (16/52), (16/53), (16/54), (16/55), (16/56), (16/57), (16/58), (16/59), (16/60), (17/5), (17/6), (17/7), (17/8), (17/9), (17/10), (17/11), (17/12), (17/13), (17/14), (17/15), (17/16), (17/17), (17/18), (17/19), (17/20), (17/21), (17/22), (17/23), (17/24), (17/25), (17/26), (17/27), (17/28), (17/29), (17/30), (17/31), (17/32), (17/33), (17/34), (17/35), (17/36), (17/37), (17/38), (17/39), (17/40), (17/41), (17/42), (17/43), (17/44), (17/45), (17/46), (17/47), (17/48), (17/49), (17/50), (17/51), (17/52), (17/53), (17/54), (17/55), (17/56), (17/57), (17/58), (17/59), (17/60), (18/5), (18/6), (18/7), (18/8), (18/9), (18/10), (18/11), (18/12), (18/13), (18/14), (18/15), (18/16), (18/17), (18/18), (18/19), (18/20), (18/21), (18/22), (18/23), (18/24), (18/25), (18/26), (18/27), (18/28), (18/29), (18/30), (18/31), (18/32), (18/33), (18/34), (18/35), (18/36), (18/37), (18/38), (18/39), (18/40), (18/41), (18/42), (18/43), (18/44), (18/45), (18/46), (18/47), (18/48), (18/49), (18/50), (18/51), (18/52), (18/53), (18/54), (18/55), (18/56), (18/57), (18/58), (18/59), (18/60), (19/5), (19/6), (19/7), (19/8), (19/9), (19/10), (19/11), (19/12), (19/13), (19/14), (19/15), (19/16), (19/17), (19/18), (19/19), (19/20), (19/21), (19/22), (19/23), (19/24), (19/25), (19/26), (19/27), (19/28), (19/29), (19/30), (19/31), (19/32), (19/33), (19/34), (19/35), (19/36), (19/37), (19/38), (19/39), (19/40), (19/41), (19/42), (19/43), (19/44), (19/45), (19/46), (19/47), (19/48), (19/49), (19/50), (19/51), (19/52), (19/53), (19/54), (19/55), (19/56), (19/57), (19/58), (19/59), (19/60), (20/5), (20/6), (20/7), (20/8), (20/9), (20/10), (20/11), (20/12), (20/13), (20/14), (20/15), (20/16), (20/17), (20/18), (20/19), (20/20), (20/21), (20/22), (20/23), (20/24), (20/25), (20/26), (20/27), (20/28), (20/29), (20/30), (20/31), (20/32), (20/33), (20/34), (20/35), (20/36), (20/37), (20/38), (20/39), (20/40), (20/41), (20/42), (20/43), (20/44), (20/45), (20/46), (20/47), (20/48), (20/49), (20/50), (20/51), (20/52), (20/53), (20/54), (20/55), (20/56), (20/57), (20/58), (20/59), (20/60), (21/5), (21/6), (21/7), (21/8), (21/9), (21/10), (21/11), (21/12), (21/13), (21/14), (21/15), (21/16), (21/17), (21/18), (21/19), (21/20), (21/21), (21/22), (21/23), (21/24), (21/25), (21/26), (21/27), (21/28), (21/29), (21/30), (21/31), (21/32), (21/33), (21/34), (21/35), (21/36), (21/37), (21/38), (21/39), (21/40), (21/41), (21/42), (21/43), (21/44), (21/45), (21/46), (21/47), (21/48), (21/49), (21/50), (21/51), (21/52), (21/53), (21/54), (21/55), (21/56), (21/57), (21/58), (21/59), (21/60), (22/5), (22/6), (22/7), (22/8), (22/9), (22/10), (22/11), (22/12), (22/13), (22/14), (22/15), (22/16), (22/17), (22/18), (22/19), (22/20), (22/21), (22/22), (22/23), (22/24), (22/25), (22/26), (22/27), (22/28), (22/29), (22/30), (22/31), (22/32), (22/33), (22/34), (22/35), (22/36), (22/37), (22/38), (22/39), (22/40), (22/41), (22/42), (22/43), (22/44), (22/45), (22/46), (22/47), (22/48), (22/49), (22/50), (22/51), (22/52), (22/53), (22/54), (22/55), (22/56), (22/57), (22/58), (22/59), (22/60), (23/5), (23/6), (23/7), (23/8), (23/9), (23/10), (23/11), (23/12), (23/13), (23/14), (23/15), (23/16), (23/17), (23/18), (23/19), (23/20), (23/21), (23/22), (23/23), (23/24), (23/25), (23/26), (23/27), (23/28), (23/29), (23/30), (23/31), (23/32), (23/33), (23/34), (23/35), (23/36), (23/37), (23/38), (23/39), (23/40), (23/41), (23/42), (23/43), (23/44), (23/45), (23/46), (23/47), (23/48), (23/49), (23/50), (23/51), (23/52), (23/53), (23/54), (23/55), (23/56), (23/57), (23/58), (23/59), (23/60), (24/5), (24/6), (24/7), (24/8), (24/9), (24/10), (24/11), (24/12), (24/13), (24/14), (24/15), (24/16), (24/17), (24/18), (24/19), (24/20), (24/21), (24/22), (24/23), (24/24), (24/25), (24/26), (24/27), (24/28), (24/29), (24/30), (24/31), (24/32), (24/33), (24/34), (24/35), (24/36), (24/37), (24/38), (24/39), (24/40), (24/41), (24/42), (24/43), (24/44), (24/45), (24/46), (24/47), (24/48), (24/49), (24/50), (24/51), (24/52), (24/53), (24/54), (24/55), (24/56), (24/57), (24/58), (24/59), (24/60), (25/5), (25/6), (25/7), (25/8), (25/9), (25/10), (25/11), (25/12), (25/13), (25/14), (25/15), (25/16), (25/17), (25/18), (25/19), (25/20), (25/21), (25/22), (25/23), (25/24), (25/25), (25/26), (25/27), (25/28), (25/29), (25/30), (25/31), (25/32), (25/33), (25/34), (25/35), (25/36), (25/37), (25/38), (25/39), (25/40), (25/41), (25/42), (25/43), (25/44), (25/45), (25/46), (25/47), (25/48), (25/49), (25/50), (25/51), (25/52), (25/53), (25/54), (25/55), (25/56), (25/57), (25/58), (25/59), (25/60), (26/5), (26/6), (26/7), (26/8), (26/9), (26/10), (26/11), (26/12), (26/13), (26/14), (26/15), (26/16), (26/17), (26/18), (26/19), (26/20), (26/21), (26/22), (26/23), (26/24), (26/25), (26/26), (26/27), (26/28), (26/29), (26/30), (26/31), (26/32), (26/33), (26/34), (26/35), (26/36), (26/37), (26/38), (26/39), (26/40), (26/41), (26/42), (26/43), (26/44), (26/45), (26/46), (26/47), (26/48), (26/49), (26/50), (26/51), (26/52), (26/53), (26/54), (26/55), (26/56), (26/57), (26/58), (26/59), (26/60), (27/5), (27/6), (27/7), (27/8), (27/9), (27/10), (27/11), (27/12), (27/13), (27/14), (27/15), (27/16), (27/17), (27/18), (27/19), (27/20), (27/21), (27/22), (27/23), (27/24), (27/25), (27/26), (27/27), (27/28), (27/29), (27/30), (27/31), (27/32), (27/33), (27/34), (27/35), (27/36), (27/37), (27/38), (27/39), (27/40), (27/41), (27/42), (27/43), (27/44), (27/45), (27/46), (27/47), (27/48), (27/49), (27/50), (27/51), (27/52), (27/53), (27/54), (27/55), (27/56), (27/57), (27/58), (27/59), (27/60), (28/5), (28/6), (28/7), (28/8), (28/9), (28/10), (28/11), (28/12), (28/13), (28/14), (28/15), (28/16), (28/17), (28/18), (28/19), (28/20), (28/21), (28/22), (28/23), (28/24), (28/25), (28/26), (28/27), (28/28), (28/29), (28/30), (28/31), (28/32), (28/33), (28/34), (28/35), (28/36), (28/37), (28/38), (28/39), (28/40), (28/41), (28/42), (28/43), (28/44), (28/45), (28/46), (28/47), (28/48), (28/49), (28/50), (28/51), (28/52), (28/53), (28/54), (28/55), (28/56), (28/57), (28/58), (28/59), (28/60), (29/5), (29/6), (29/7), (29/8), (29/9), (29/10), (29/11), (29/12), (29/13), (29/14), (29/15), (29/16), (29/17), (29/18), (29/19), (29/20), (29/21), (29/22), (29/23), (29/24), (29/25), (29/26), (29/27), (29/28), (29/29), (29/30), (29/31), (29/32), (29/33), (29/34), (29/35), (29/36), (29/37), (29/38), (29/39), (29/40), (29/41), (29/42), (29/43), (29/44), (29/45), (29/46), (29/47), (29/48), (29/49), (29/50), (29/51), (29/52), (29/53), (29/54), (29/55), (29/56), (29/57), (29/58), (29/59), (29/60), (30/5), (30/6), (30/7), (30/8), (30/9), (30/10), (30/11), (30/12), (30/13), (30/14), (30/15), (30/16), (30/17), (30/18), (30/19), (30/20), (30/21), (30/22), (30/23), (30/24), (30/25), (30/26), (30/27), (30/28), (30/29), (30/30), (30/31), (30/32), (30/33), (30/34), (30/35), (30/36), (30/37), (30/38), (30/39), (30/40), (30/41), (30/42), (30/43), (30/44), (30/45), (30/46), (30/47), (30/48), (30/49), (30/50), (30/51), (30/52), (30/53), (30/54), (30/55), (30/56), (30/57), (30/58), (30/59), (30/60), (31/5), (31/6), (31/7), (31/8), (31/9), (31/10), (31/11), (31/12), (31/13), (31/14), (31/15), (31/16), (31/17), (31/18), (31/19), (31/20), (31/21), (31/22), (31/23), (31/24), (31/25), (31/26), (31/27), (31/28), (31/29), (31/30), (31/31), (31/32), (31/33), (31/34), (31/35), (31/36), (31/37), (31/38), (31/39), (31/40), (31/41), (31/42), (31/43), (31/44), (31/45), (31/46), (31/47), (31/48), (31/49), (31/50), (31/51), (31/52), (31/53), (31/54), (31/55), (31/56), (31/57), (31/58), (31/59), (31/60), (32/5), (32/6), (32/7), (32/8), (32/9), (32/10), (32/11), (32/12), (32/13), (32/14), (32/15), (32/16), (32/17), (32/18), (32/19), (32/20), (32/21), (32/22), (32/23), (32/24), (32/25), (32/26), (32/27), (32/28), (32/29), (32/30), (32/31), (32/32), (32/33), (32/34), (32/35), (32/36), (32/37), (32/38), (32/39), (32/40), (32/41), (32/42), (32/43), (32/44), (32/45), (32/46), (32/47), (32/48), (32/49), (32/50), (32/51), (32/52), (32/53), (32/54), (32/55), (32/56), (32/57), (32/58), (32/59), (32/60), (33/5), (33/6), (33/7), (33/8), (33/9), (33/10), (33/11), (33/12), (33/13), (33/14), (33/15), (33/16), (33/17), (33/18), (33/19), (33/20), (33/21), (33/22), (33/23), (33/24), (33/25), (33/26), (33/27), (33/28), (33/29), (33/30), (33/31), (33/32), (33/33), (33/34), (33/35), (33/36), (33/37), (33/38), (33/39), (33/40), (33/41), (33/42), (33/43), (33/44), (33/45), (33/46), (33/47), (33/48), (33/49), (33/50), (33/51), (33/52), (33/53), (33/54), (33/55), (33/56), (33/57), (33/58), (33/59), (33/60), (34/5), (34/6), (34/7), (34/8), (34/9), (34/10), (34/11), (34/12), (34/13), (34/14), (34/15), (34/16), (34/17), (34/18), (34/19), (34/20), (34/21), (34/22), (34/23), (34/24), (34/25), (34/26), (34/27), (34/28), (34/29), (34/30), (34/31), (34/32), (34/33), (34/34), (34/35), (34/36), (34/37), (34/38), (34/39), (34/40), (34/41), (34/42), (34/43), (34/44), (34/45), (34/46), (34/47), (34/48), (34/49), (34/50), (34/51), (34/52), (34/53), (34/54), (34/55), (34/56), (34/57), (34/58), (34/59), (34/60), (35/5), (35/6), (35/7), (35/8), (35/9), (35/10), (35/11), (35/12), (35/13), (35/14), (35/15), (35/16), (35/17), (35/18), (35/19), (35/20), (35/21), (35/22), (35/23), (35/24), (35/25), (35/26), (35/27), (35/28), (35/29), (35/30), (35/31), (35/32), (35/33), (35/34), (35/35), (35/36), (35/37), (35/38), (35/39), (35/40), (35/41), (35/42), (35/43), (35/44), (35/45), (35/46), (35/47), (35/48), (35/49), (35/50), (35/51), (35/52), (35/53), (35/54), (35/55), (35/56), (35/57), (35/58), (35/59), (35/60), (36/5), (36/6), (36/7), (36/8), (36/9), (36/10), (36/11), (36/12), (36/13), (36/14), (36/15), (36/16), (36/17), (36/18), (36/19), (36/20), (36/21), (36/22), (36/23), (36/24), (36/25), (36/26), (36/27), (36/28), (36/29), (36/30), (36/31), (36/32), (36/33), (36/34), (36/35), (36/36), (36/37), (36/38), (36/39), (36/40), (36/41), (36/42), (36/43), (36/44), (36/45), (36/46), (36/47), (36/48), (36/49), (36/50), (36/51), (36/52), (36/53), (36/54), (36/55), (36/56), (36/57), (36/58), (36/59), (36/60), (37/5), (37/6), (37/7), (37/8), (37/9), (37/10), (37/11), (37/12), (37/13), (37/14), (37/15), (37/16), (37/17), (37/18), (37/19), (37/20), (37/21), (37/22), (37/23), (37/24), (37/25), (37/26), (37/27), (37/28), (37/29), (37/30), (37/31), (37/32), (37/33), (37/34), (37/35), (37/36), (37/37), (37/38), (37/39), (37/40), (37/41), (37/42), (37/43), (37/44), (37/45), (37/46), (37/47), (37/48), (37/49), (37/50), (37/51), (37/52), (37/53), (37/54), (37/55), (37/56), (37/57), (37/58), (37/59), (37/60), (38/5), (38/6), (38/7), (38/8), (38/9), (38/10), (38/11), (38/12), (38/13), (38/14), (38/15), (38/16), (38/17), (38/18), (38/19), (38/20), (38/21), (38/22), (38/23), (38/24), (38/25), (38/26), (38/27), (38/28), (38/29), (38/30), (38/31), (38/32), (38/33), (38/34), (38/35), (38/36), (38/37), (38/38), (38/39), (38/40), (38/41), (38/42), (38/43), (38/44), (38/45), (38/46), (38/47), (38/48), (38/49), (38/50), (38/51), (38/52), (38/53), (38/54), (38/55), (38/56), (38/57), (38/58), (38/59), (38/60), (39/5), (39/6), (39/7), (39/8), (39/9), (39/10), (39/11), (39/12), (39/13), (39/14), (39/15), (39/16), (39/17), (39/18), (39/19), (39/20), (39/21), (39/22), (39/23), (39/24), (39/25), (39/26), (39/27), (39/28), (39/29), (39/30), (39/31), (39/32), (39/33), (39/34), (39/35), (39/36), (39/37), (39/38), (39/39), (39/40), (39/41), (39/42), (39/43), (39/44), (39/45), (39/46), (39/47), (39/48), (39/49), (39/50), (39/51), (39/52), (39/53), (39/54), (39/55), (39/56), (39/57), (39/58), (39/59), (39/60), (40/5), (40/6), (40/7), (40/8), (40/9), (40/10), (40/11), (40/12), (40/13), (40/14), (40/15), (40/16), (40/17), (40/18), (40/19), (40/20), (40/21), (40/22), (40/23), (40/24), (40/25), (40/26), (40/27), (40/28), (40/29), (40/30), (40/31), (40/32), (40/33), (40/34), (40/35), (40/36), (40/37), (40/38), (40/39), (40/40), (40/41), (40/42), (40/43), (40/44), (40/45), (40/46), (40/47), (40/48), (40/49), (40/50), (40/51), (40/52), (40/53), (40/54), (40/55), (40/56), (40/57), (40/58), (40/59), (40/60), (41/5), (41/6), (41/7), (41/8), (41/9), (41/10), (41/11), (41/12), (41/13), (41/14), (41/15), (41/16), (41/17), (41/18), (41/19), (41/20), (41/21), (41/22), (41/23), (41/24), (41/25), (41/26), (41/27), (41/28), (41/29), (41/30), (41/31), (41/32), (41/33), (41/34), (41/35), (41/36), (41/37), (41/38), (41/39), (41/40), (41/41), (41/42), (41/43), (41/44), (41/45), (41/46), (41/47), (41/48), (41/49), (41/50), (41/51), (41/52), (41/53), (41/54), (41/55), (41/56), (41/57), (41/58), (41/59), (41/60), (42/5), (42/6), (42/7), (42/8), (42/9), (42/10), (42/11), (42/12), (42/13), (42/14), (42/15), (42/16), (42/17), (42/18), (42/19), (42/20), (42/21), (42/22), (42/23), (42/24), (42/25), (42/26), (42/27), (42/28), (42/29), (42/30), (42/31), (42/32), (42/33), (42/34), (42/35), (42/36), (42/37), (42/38), (42/39), (42/40), (42/41), (42/42), (42/43), (42/44), (42/45), (42/46), (42/47), (42/48), (42/49), (42/50), (42/51), (42/52), (42/53), (42/54), (42/55), (42/56), (42/57), (42/58), (42/59), (42/60), (43/5), (43/6), (43/7), (43/8), (43/9), (43/10), (43/11), (43/12), (43/13), (43/14), (43/15), (43/16), (43/17), (43/18), (43/19), (43/20), (43/21), (43/22), (43/23), (43/24), (43/25), (43/26), (43/27), (43/28), (43/29), (43/30), (43/31), (43/32), (43/33), (43/34), (43/35), (43/36), (43/37), (43/38), (43/39), (43/40), (43/41), (43/42), (43/43), (43/44), (43/45), (43/46), (43/47), (43/48), (43/49), (43/50), (43/51), (43/52), (43/53), (43/54), (43/55), (43/56), (43/57), (43/58), (43/59), (43/60), (44/5), (44/6), (44/7), (44/8), (44/9), (44/10), (44/11), (44/12), (44/13), (44/14), (44/15), (44/16), (44/17), (44/18), (44/19), (44/20), (44/21), (44/22), (44/23), (44/24), (44/25), (44/26), (44/27), (44/28), (44/29), (44/30), (44/31), (44/32), (44/33), (44/34), (44/35), (44/36), (44/37), (44/38), (44/39), (44/40), (44/41), (44/42), (44/43), (44/44), (44/45), (44/46), (44/47), (44/48), (44/49), (44/50), (44/51), (44/52), (44/53), (44/54), (44/55), (44/56), (44/57), (44/58), (44/59), (44/60), (45/5), (45/6), (45/7), (45/8), (45/9), (45/10), (45/11), (45/12), (45/13), (45/14), (45/15), (45/16), (45/17), (45/18), (45/19), (45/20), (45/21), (45/22), (45/23), (45/24), (45/25), (45/26), (45/27), (45/28), (45/29), (45/30), (45/31), (45/32), (45/33), (45/34), (45/35), (45/36), (45/37), (45/38), (45/39), (45/40), (45/41), (45/42), (45/43), (45/44), (45/45), (45/46), (45/47), (45/48), (45/49), (45/50), (45/51), (45/52), (45/53), (45/54), (45/55), (45/56), (45/57), (45/58), (45/59), (45/60), (46/5), (46/6), (46/7), (46/8), (46/9), (46/10), (46/11), (46/12), (46/13), (46/14), (46/15), (46/16), (46/17), (46/18), (46/19), (46/20), (46/21), (46/22), (46/23), (46/24), (46/25), (46/26), (46/27), (46/28), (46/29), (46/30), (46/31), (46/32), (46/33), (46/34), (46/35), (46/36), (46/37), (46/38), (46/39), (46/40), (46/41), (46/42), (46/43), (46/44), (46/45), (46/46), (46/47), (46/48), (46/49), (46/50), (46/51), (46/52), (46/53), (46/54), (46/55), (46/56), (46/57), (46/58), (46/59), (46/60), (47/5), (47/6), (47/7), (47/8), (47/9), (47/10), (47/11), (47/12), (47/13), (47/14), (47/15), (47/16), (47/17), (47/18), (47/19), (47/20), (47/21), (47/22), (47/23), (47/24), (47/25), (47/26), (47/27), (47/28), (47/29), (47/30), (47/31), (47/32), (47/33), (47/34), (47/35), (47/36), (47/37), (47/38), (47/39), (47/40), (47/41), (47/42), (47/43), (47/44), (47/45), (47/46), (47/47), (47/48), (47/49), (47/50), (47/51), (47/52), (47/53), (47/54), (47/55), (47/56), (47/57), (47/58), (47/59), (47/60), (48/5), (48/6), (48/7), (48/8), (48/9), (48/10), (48/11), (48/12), (48/13), (48/14), (48/15), (48/16), (48/17), (48/18), (48/19), (48/20), (48/21), (48/22), (48/23), (48/24), (48/25), (48/26), (48/27), (48/28), (48/29), (48/30), (48/31), (48/32), (48/33), (48/34), (48/35), (48/36), (48/37), (48/38), (48/39), (48/40), (48/41), (48/42), (48/43), (48/44), (48/45), (48/46), (48/47), (48/48), (48/49), (48/50), (48/51), (48/52), (48/53), (48/54), (48/55), (48/56), (48/57), (48/58), (48/59), (48/60), (49/5), (49/6), (49/7), (49/8), (49/9), (49/10), (49/11), (49/12), (49/13), (49/14), (49/15), (49/16), (49/17), (49/18), (49/19), (49/20), (49/21), (49/22), (49/23), (49/24), (49/25), (49/26), (49/27), (49/28), (49/29), (49/30), (49/31), (49/32), (49/33), (49/34), (49/35), (49/36), (49/37), (49/38), (49/39), (49/40), (49/41), (49/42), (49/43), (49/44), (49/45), (49/46), (49/47), (49/48), (49/49), (49/50), (49/51), (49/52), (49/53), (49/54), (49/55), (49/56), (49/57), (49/58), (49/59), (49/60), (50/5), (50/6), (50/7), (50/8), (50/9), (50/10), (50/11), (50/12), (50/13), (50/14), (50/15), (50/16), (50/17), (50/18), (50/19), (50/20), (50/21), (50/22), (50/23), (50/24), (50/25), (50/26), (50/27), (50/28), (50/29), (50/30), (50/31), (50/32), (50/33), (50/34), (50/35), (50/36), (50/37), (50/38), (50/39), (50/40), (50/41), (50/42), (50/43), (50/44), (50/45), (50/46), (50/47), (50/48), (50/49), (50/50), (50/51), (50/52), (50/53), (50/54), (50/55), (50/56), (50/57), (50/58), (50/59), or (50/60).

In contrast to the triblock surfactant 600, the di-block surfactant 700 comprises only one fluorophilic tail 704, which is a PFPE chain linked to the hydrophilic head group 702, a PEG group, via an amide bond.

The fluorophilic tail or fluorophilic component of surfactant molecules of Formula II described herein may comprise a fluorophilic chain at least C₈ in length (i.e., contains at least 8 carbon atoms). In some cases, the fluorophilic chain may be at least C₁₀ in length, at least C₁₅ in length, at least C₂₀ in length, at least C₂₅ in length, or at least C₃₀ in length. In other cases, the fluorophilic chain may be at least C₅₀ in length, at least C₇₅ in length, at least C₁₀₀ length, or greater than 100 carbon atoms. As a non-limiting example, a fluorophilic component having the structure —(C₃F6O)₁₀— has 30 carbons equivalent to a C₃₀ chain. The fluorophilic component may be linear, branched, cyclic, saturated, unsaturated, etc. In some case, the fluorophilic component of a surfactant may be a fluorinated oligomer or polymer (i.e., a fluoropolymer). The fluoropolymer may include a perfluoropolyether chain, among other fluorinated polymers that are soluble in a fluorocarbon oil. The fluorophilic tail of a surfactant may have any suitable mixture of hydrogen and fluorine atoms so long as the fluorophilic component is soluble in a suitable fluorophilic continuous phase to permit the subsequent emulsion formation.

The fluorophilic component may have a molecular weight greater than or equal to 500 g/mol, greater than or equal to 800 g/mol, greater than or equal to 1,000 g/mol, greater than or equal to 1,200 g/mol, greater than or equal to 1,500 g/mol, greater than or equal to 1,700 g/mol, greater than or equal to 1,900 g/mol, greater than or equal to 2,000 g/mol, greater than or equal to 2,200 g/mol, greater than or equal to 2,500 g/mol, greater than or equal to 2,700 g/mol, greater than or equal to 3,000 g/mol, greater than or equal to 3,200 g/mol, greater than or equal to 3,500 g/mol, greater than or equal to 3,700 g/mol, greater than or equal to 4,000 g/mol, greater than or equal to 4,200 g/mol, greater than or equal to 4,500 g/mol, greater than or equal to 4,700 g/mol, greater than or equal to 5,000 g/mol, greater than or equal to 5,200 g/mol, greater than or equal to 5,500 g/mol, greater than or equal to 5,700 g/mol, greater than or equal to 6,000 g/mol, greater than or equal to 6,200 g/mol, greater than or equal to 6,500 g/mol, greater than or equal to 6,700 g/mol, greater than or equal to 7,000 g/mol, greater than or equal to 7,200 g/mol, greater than or equal to 7,500 g/mol, greater than or equal to 7,700 g/mol, greater than or equal to 8,000 g/mol, greater than or equal to 8,200 g/mol, greater than or equal to 8,500 g/mol, greater than or equal to 8,700 g/mol, greater than or equal to 9,000 g/mol, greater than or equal to 9,200 g/mol, greater than or equal to 9,500 g/mol, greater than or equal to 9,700 g/mol, or greater than or equal to 10,000 g/mol.

When the di-block surfactant, such as, for example, compounds of Formula II, is used to form emulsion droplets, the concentration of the di-block surfactant may be about 0.1 mM, about 0.2 mM, about 0.3 mM, about 0.5 mM, about 0.6 mM, about 0.7 mM, about 0.8 mM, about 0.9 mM, about 1.0 mM, about 1.1 mM, about 1.2 mM, about 1.3 mM, 1.4 mM, about 1.5 mM, about 1.6 mM, about 1.7 mM, about 1.8 mM, about 1.9 mM, about 2.0 mM, about 2.1 mM, about 2.2 mM, about 2.3 mM, 2.4 mM, about 2.5 mM, about 2.6 mM, about 2.7 mM, about 2.8 mM, about 2.9 mM, about 3.0 mM, about 3.1 mM, about 3.2 mM, about 3.3 mM, 3.4 mM, about 3.5 mM, about 3.6 mM, about 3.7 mM, about 3.8 mM, about 3.9 mM, about 4.0 mM, about 4.5 mM, about 5.0 mM, about 6.0 mM, 7.0 mM, about 8.0 mM, about 9.0 mM, about 10 mM, about 20 mM, about 30 mM, about 40 mM, about 50 mM, about 60 mM, about 70 mM, about 80 mM, about 90 mM, or about 100 mM.

When the di-block surfactant, such as, for example, compounds of Formula II, is used to form emulsion droplets, the percentage of the emulsion droplets that coalesced is at most 1.0%, at most 1.5%, at most 2.0%, at most 2.5%, at most 3.0%, at most 3.5%, at most 4.0%, at most 4.5%, at most 5.0%, at most 5.5%, at most 6.0%, at most 6.5%, at most 7.0%, at most 7.5%, at most 8.0%, at most 8.5%, at most 9.0%, at most 10%, at most 15%, or at most 20%.

When the di-block surfactant, such as, for example, compounds of Formula II, is used to form emulsion droplets, the percentage of the emulsion droplets that coalesced due to surface-mediated coalescence is at most 1.0%, at most 1.5%, at most 2.0%, at most 2.5%, at most 3.0%, at most 3.5%, at most 4.0%, at most 4.5%, at most 5.0%, at most 5.5%, at most 6.0%, at most 6.5%, at most 7.0%, at most 7.5%, at most 8.0%, at most 8.5%, at most 9.0%, at most 10%, at most 15%, or at most 20%.

When the di-block surfactant, such as, for example, compounds of Formula II, is used to form emulsion droplets and a lyse agent, such as, for example, n-dodecyl-β-D-maltoside (DBDM), is used inside the emulsion droplets, the percentage of the emulsion droplets that coalesced due to surface-mediated coalescence is at most 1.0%, at most 1.5%, at most 2.0%, at most 2.5%, at most 3.0%, at most 3.5%, at most 4.0%, at most 4.5%, at most 5.0%, at most 5.5%, at most 6.0%, at most 6.5%, at most 7.0%, at most 7.5%, at most 8.0%, at most 8.5%, at most 9.0%, at most 10%, at most 15%, or at most 20%.

A synthetic route leading to the synthesis of compounds of Formula II is shown in Scheme 1:

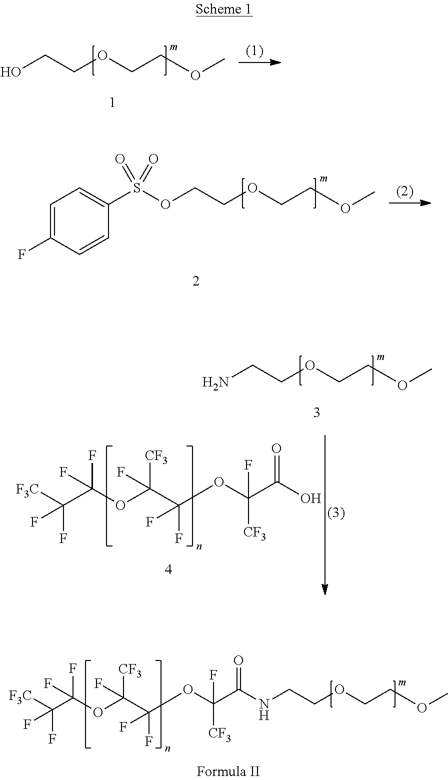

Here, m is an integer from 5 to 50, preferably from 10 to 30, more preferably from 16 to 22; n is an integer 5 to 60, preferably from 20 to 50, more preferably from 33 to 39. Examples of conditions for reactions depicted in Scheme 1 (when starting with about 40 g of poly(ethylene glycol) methyl ether 1, 1.0 equivalent (eq.)) are: (1) i) NaH (1.5 eq.), THF (150 mL), room temperature, 5 hours, ii) 4-fluorobenzensulfonyl chloride (FTsCl, 1.1 eq.), THF (200 m), room temperature, under Argon (Ar.), overnight (about 16 hours); (2) NH₄OH (300 mL), room temperature, overnight (about 16 hours); and (3) i) oxalyl chloride (4.0 eq.), DMF (0.04 eq.), the carboxylic acid reactant 4 (1.0 eq.) in hydrofluoroether (HFE) such as HFE-7100 (methoxy-nonafluorobutane, 75 mL), reflux (at about 60° C.), 16 hours, under Ar., ii) the amine reagent 3 (1.1 eq.) and Et₃N (2.0 eq.), reflux (at about 60° C.) in HFE-7100 (75 mL)/THF (30 mL), overnight (about 16 hours).

For the tosylation step (1), a stoichiometry of about 1.0 equivalent (eq.) of mono-methyl protected PEG (MPEG) reagent 1, about 1.5 eq. NaH, and about 1.1 eq. of FTsCl affords the production of the expected MPEG tosylate 2 with a reduced amount of FTsCl leftovers in the final product when compared with other conditions when excess amount of FTsCl is used. In some cases, the tosylation reaction may give about 93% yield for the products with about 10% unreacted FTsCl as an impurity.

The ensuing amination followed by recrystallization in about 3 hours gives about 70% yield for the expected amine 3 with about 2% FTsCl impurities. Coupling with an activated perfluoro-acyl chloride reagent 4 affords the di-block surfactant of Formula II in about 80% conversion rates. Unreacted non-fluorous materials are removed during work up with fluorous solvent. For the coupling step (3), a stoichiometry of about 1.0 eq. of carboxylic acid 4, about 4.0 eq. of oxalyl chloride with about 0.04 eq. of DMF for sub-step i), followed by about 1.1 eq. of amine 3 and about 2.0 eq. of Et₃N in sub-step ii), produces the desired amide. The surfactant syntheses described herein may employ a perfluorinated compound or polymer, such as a poly(perfluoro-propyleneoxide) (e.g. KRYTOX® by DuPont).

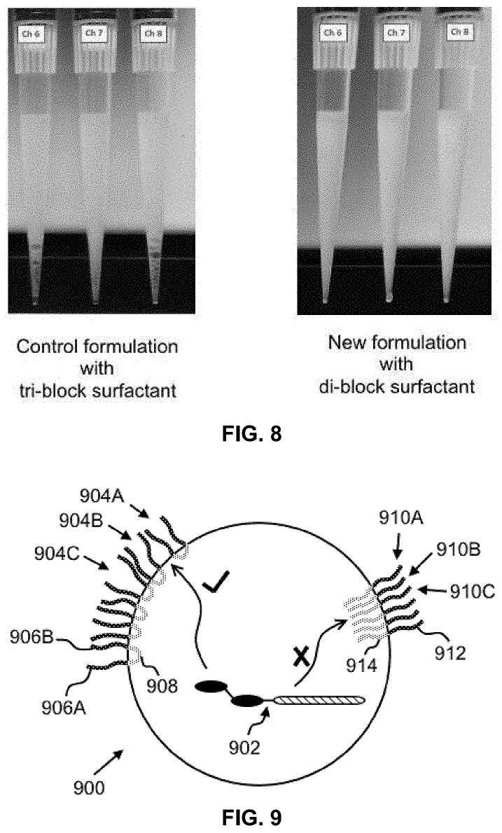

Replacing the tri-block surfactant with the di-block surfactant, such as, for example, compounds of Formula II, reduces the extent of surface-mediated coalescence while keeping other components and emulsion-forming conditions the same. Emulsions may be generated using a 10× Chromium controller employing the 10× Single Cell 3′ procedure and reagents. FIG. 8 shows the results when one formulation uses tri-block surfactant and another formulation uses di-block surfactant. As shown in FIG. 8, emulsions in the left panel uses the tri-block surfactant-based formulation while emulsions in the right panel uses the di-block surfactant-based formulation. Channels 6-8 of the left panel exhibit surface-mediated coalescence in the presence of the tri-block surfactant at the bottom of the pipettes, similar to that shown in FIG. 4. In contract, channels 6-8 of the right panel do not show surface-mediated coalescence in the presence of the di-block surfactant. As disclosed herein, the instrument, conditions and components other than the surfactant used may be kept the same for the experiments shown in FIG. 8. In both experiments, DBDM is used as the lysis agent.

The stabilization of droplets in a fluorophilic continuous phase may involve the factors and criteria described below or elsewhere herein. As shown in the diagram illustrated in the left side of FIG. 9, an emulsion droplet/micelle 900 may comprise lysis agent DBDM 902 inside its aqueous phase and a plurality of tri-block surfactants 904, including, for example, 904A, 904B, and 904C, forming a barrier at the interface between the fluorocarbon oil phase and the aqueous phase during the emulsification. Each tri-block surfactants 904 may comprise two fluorophilic tails 906A and 906B and one hydrophilic head group 908, as shown in FIG. 9. The two fluorophilic tails 906A and 906B may point toward exterior of the micelle 900 by bending the hydrophilic head group 908, which is the linker between the two fluorophilic tails 906A and 906B. In such a conformation, neighboring fluorophilic tails 906 may bump into each other due to the strains created by the bending hydrophilic head group 908, thereby creating a gap between the tri-block surfactants 904A and 904B as shown in FIG. 9. Further, a roughened surface may push the neighboring fluorophilic tails 906 in such a way that may work in sync with or on top of the existing tension caused by the bending hydrophilic head group 908 in this conformation, thereby pushing the gap even further between the tri-block surfactants 904A and 904B. Finally, the lysis agent DBDM 902 may be capable of disrupting the surfactant barrier formed from tri-block surfactants 904. For example, the lysis agent DBDM 902 may move like a wedge to insert itself through the gap between the tri-block surfactants 904A and 904B, thereby further widening the gap to break the micelle. Surface-mediated coalescence may occur with tri-block surfactants and the lysis agent such as DBDM in the presence of a roughened surface.

In contrast, as shown in in the right side of FIG. 9, an emulsion droplet/micelle 900 may comprise lysis agent DBDM 902 and a plurality of new di-block surfactants 910, including, for example, 910A, 910B, and 910C, forming a barrier at the interface between the fluorocarbon oil phase and the aqueous phase during emulsification. Each new di-block surfactants 910 may comprise one fluorophilic tail 912 and one hydrophilic head group 914, as shown in FIG. 9. The fluorophilic tail 912 may point toward the exterior of the micelle 900 and the hydrophilic head group 914 may point toward the interior of the micelle 900. Because all hydrophilic head groups 914 point toward the interior of the micelle 900 in this conformation, there may be much less strain for the new di-block surfactants 910 when compared with the conformation of tri-block surfactants 904 described above. Furthermore, in the case of tri-block surfactant 904, the bending hydrophilic head group 908 may form an arc near the surface barrier. As a result, this arc may lead to less surface packing density with respect to the fluorophilic tails 906A and 906B since they are prevented from coming closer by the arc formed from the bending hydrophilic head group 909. In the case of the di-block surfactant 910, there may not be such arc-required separation. Accordingly, the new di-block surfactants 910 may pack more densely than tri-block surfactants 904. This higher surface packing efficient may strengthen the resistance of the barrier formed by the new di-block surfactants 910 toward both the penetration by the lysis agent DBDM and the disturbance caused by the roughened surface associated during the process of coalescence. Surface-mediated coalescence in the presence of DBDM 902 may occur to a smaller extent with di-block surfactants 910 than with tri-block surfactants 904.

### Computer Control System

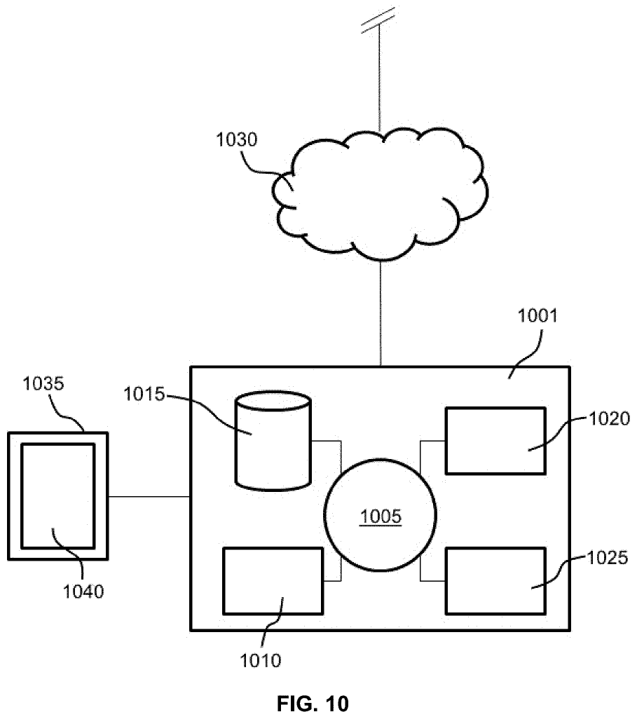

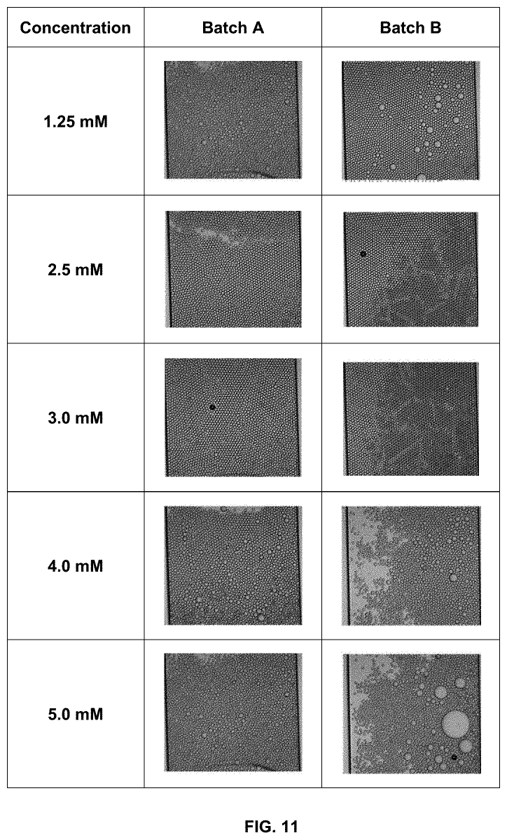

The present disclosure provides computer control systems that are programmed to implement methods of the disclosure. FIG. 10 shows a computer system 1001 that is programmed or otherwise configured to implement methods of the disclosure including nucleic acid sequencing methods, emulsion formation methods, interpretation of nucleic acid sequencing data and analysis of cellular nucleic acids, such as RNA (e.g., mRNA), interpretation of nucleic acid sequencing data and analysis of nucleic acids derived from the characterization of cellular nucleic acids, and characterization of cells from sequencing data. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, samples and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., APPLE® iPad, SAMSUNG® Galaxy Tab), telephones, Smart phones (e.g., APPLE® iPhone, Android-enabled device, BLACKBERRY®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” generally refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. Shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, results of nucleic acid sequencing, analysis of nucleic acid sequencing data, characterization of nucleic acid sequencing samples, cell characterizations, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, monitor and change reaction conditions, initiate nucleic acid sequencing, process nucleic acid sequencing data, interpret nucleic acid sequencing results, characterize nucleic acid samples, etc.

### EXAMPLE 1: TOSYLATION PROCEDURE

The following is an example of a procedure to form a tosylate: A mixture of monomethylether polyethylene glycol (MPEG) reagent 1 (40 g, 1.0 eq.) and NaH (1.5 eq.) in tetrahydrofuran (THF) (150 mL) is kept under argon and stirred for 5 hours (h). In another flask 4-fluorobenzensulfonyl chloride (FTsCl) (1.1 eq.) is dissolved in 30 mL of THF. The previously made MPEG-alkoxide solution is then transferred into the FTsCl solution over 1 hour. After the transfer is completed, the resulting mixture is stirred at room temperature for 16 hours. Then the reaction mixture is filtered three times to remove solid materials and the final filtrate is evaporated to dryness to afford the desired tosylate 2 together with some unreacted FTsCl.

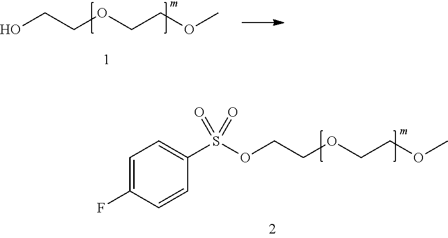

### EXAMPLE 2: AMINATION PROCEDURE

Tosylate 2 obtained in Example 1 is dissolved in ammonium hydroxide (300 ml, about 29% ammonia) and the reaction mixture is stirred constantly at room temperature for 16 hours. Then the mixture is extracted with CH₂Cl₂ (DCM, 80 mL×3) and the combined organic layers are washed once with a saturated NaCl solution (300 mL). The resulting organic layer is dried with anhydrous MgSO₄, filtered, and concentrated. The residue is recrystallized in CH₂Cl₂ (10 mL)-Et₂O (300 mL) mixture at −20° C. to afford the desired amine 3.

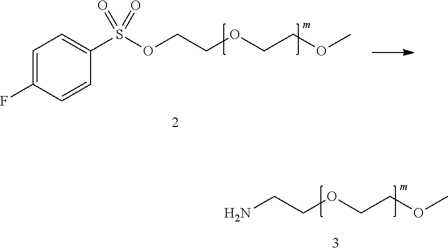

### EXAMPLE 3: ACYLATION PROCEDURE

Perfluorinated carboxylic acid 4 (Krytox 157 FS(H), 150 g, 1.0 eq.) is treated with DMF (0.04 eq.) and oxalyl chloride (4.0 eq.) in HFE-7100 solvent (125 mL) at 70° C. for 5 hours. Then the reaction mixture is allowed to cool to room temperature and volatiles are removed by evaporation under argon. The resulting perfluorinated acid chloride derivative is dissolved in dry HFE 7100 (125 mL) under argon. A solution of amine 3 obtained in Example 2 (1.2 eq.) and freshly distilled Et₃N (2.0 eq.) dissolved in THF (30 mL) is added to the perfluorinated acid chloride solution. The resulting reaction mixture is refluxed at 70° C. overnight. Then the mixture is cooled to room temperature and the mixture is evaporated to dryness. The resulting crude perfluorous product is dissolved in HFE 7100 (300 mL), transferred to a 2 L separatory funnel using additional HFE 7100 (150 mL×2) to help the transfer. After vigorous shaking, the mixture is allowed to stand for at least 4 hours inside the separatory funnel, then the mixture is filtered through a 10-20 sintered glass frit Bucher funnel. Filtrates are combined and concentrated to give the desired di-block surfactant of Formula II.

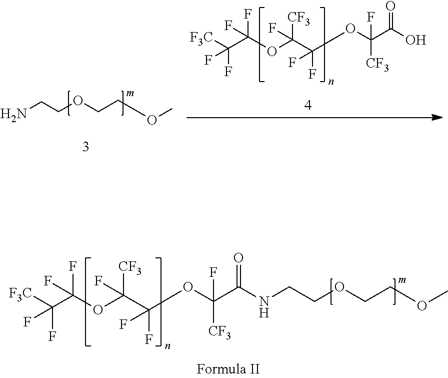

### EXAMPLE 4: SELECTING CONCENTRATION OF DI-BLOCK SURFACTANT IN OIL

Two replicate surfactant batches (Batch A and Batch B) made from synthetic routes described in Scheme 1 are formulated in HFE-7500 oil in concentrations of about 1.25 mM, about 2.5 mM, about 3.0 mM, about 4.0 mM, and about 5.0 mM, respectively. These formulations are then used to prepare droplets using an emulsion protocol on a 10× Chromium controller. Then the emulsions formed are thermally cycled according to protocol described in the 10× GEMCODE™ workflow (GEMCODE™ User Guide, Revision B, August 2015, section 5.2.3). At the end of the thermal cycles emulsion droplets for each formulation are analyzed under a microscope to determine the extent of coalescence. Snapshots for each formulation are shown in FIG. 11. FIG. 11 shows that at concentrations of about 2.5 mM and about 3.0 mM for both batches, the extent of coalescence is the least observed. Lower concentration of about 1.25 mM and higher concentrations of about 4.0 mM and about 5.0 mM all lead to more observed coalescence, according to FIG. 11.

### EXAMPLE 5: TESTING FORMULATIONS WITH 2.5 mM DI-BLOCK SURFACTANT

The above two batches of di-block surfactant-based formulations with the di-block surfactant at about 2.5 mM and a control formulation (which used the tri-block surfactant in HFE-7500) are tested for other properties, including, for example, interfacial tension, micelle size, critical micelle concentration (CMC), and viscosity. The results are summarized in Table 1 below.

TABLE 1
Properties of Batches A and B with 2.5 mM Di-Block Surfactant

| Metric | Control | | Batch A | | Batch B | |
|---|---|---|---|---|---|---|
| Interfacial | 10.0 | mN/m | 7.2 | mN/m | 6.7 | mN/m |
| Tension | | | | | | |
| Micelle Size | 177.50 | nm | 43.82 | nm | 72.09 | nm |
| CMC | 1.0-5.0 | μM | 7.5-15 | μM | 7.5-15 | μM |

According to Table 1, compared with the control formulation, the di-block surfactant-based formulations have a smaller micelle size, lower interfacial tension and higher CMC. The functional impact of lower interfacial tension may be that the di-block surfactant-based formulations ran faster in tubes or reaction chambers of analytical instruments than the control formulation based on tri-block surfactant.

### EXAMPLE 6: TESTING SINGLE CELL SEQUENCING USING THE NEW FORMULATION

A Barnyard quality control experiment is conducted using the control formulation and the new formulation described in Examples 1 and 2 on 1:1 mixtures of cultured human (293T) and mouse (3T3) cells, scoring the numbers of human and mouse transcripts that associated with each cell barcode among other metrics. Experiments are completed using commercially available 10× Single Cell 3′ workflow using a 10× Chromium controller. Results obtained are shown in Table 2 below.

TABLE 2
Results of Testing Single Cell Sequencing Using Mixtures of Cells

| | | Control | |
|---|---|---|---|
| Metric | Specification | Formulation | New Formulation |
| Number of Cells (Targeted 1,200) | 800-7,200 | 1612 | 1428 |
| Number of Reads | — | 112,440,412 | 109,129,004 |
| Mean Reads per Cell | >2,000 | 69,752 | 76,420 |
| Valid Barcodes | ≥0.75 | 0.955 | 0.956 |
| Reads Mapped Confidently to | ≥0.5 | 0.63 | 0.66 |
| Transcriptome | | | |
| hg19 Fraction Reads in Cells | ≥0.65 | 0.732 | 0.718 |
| mm10 Fraction Reads in Cells | ≥0.65 | 0.869 | 0.872 |
| Multiplet rate per 1000 cells | ≤0.02 | 0.006 | 0.013 |
| Mean UMI Count Purity | >0.98 | 0.988 | 0.989 |
| Barcodes detected | ≥90,000 | 264,958 | 321,573 |
| hg19 Median Genes per Cell | Record Only | 3678 | 3983 |
| mm10 Median Genes per Cell | Record Only | 3141 | 3458 |
| Hg19 Median Genes per cell | ≥2,290 | 2378 | 2381 |
| (20k raw reads per cell) | | | |
| mm10 Median Genes per cell | ≥1,590 | 1968 | 2047 |
| (20k raw reads per cell) | | | |

Accordingly, both the control formulation and the new formulation using di-block surfactant give similar sequencing results across multiple categories. In addition, to maintain a low coalescence rate of less than 5%, the control formulation using tri-block surfactant may require an undefined wait time between completion of formulation and the use in sequencing experiments. Such wait time may vary from about four weeks to about eight weeks and be batch-dependent. When the control formulation is used in emulsion formation within one day or within one week of the formulation, emulsions formed may be found to be unstable to the extent of over 50% coalescence, over 80% coalescence, or over 90% coalescence. In contrast, the new formulations based on di-block surfactant may be used to form emulsions on the same day of formulation and still keep the emulsions stable with less than 5% coalescence. For these experiments, emulsions are imaged under microscope and the coalescence levels are determined using a software developed to identify droplets within an image and bin each droplet according to size.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for forming an emulsion comprising a plurality of droplets, comprising:
(a) bringing a first fluid phase in contact with a second fluid phase that is immiscible with said first fluid phase to generate an emulsion comprising said plurality of droplets, wherein said plurality of droplets comprises (i) said first fluid phase or said second fluid phase, (ii) a first surfactant at an interface between said first fluid phase and said second fluid phase, and (iii) a second surfactant that is different than said first surfactant; and
(b) upon generating at least a subset of said plurality of droplets, (i) collecting said plurality of droplets or (ii) directing said plurality of droplets along a channel, wherein upon collecting or directing said plurality of droplets along said channel, at most 5% of said plurality of droplets coalesce.

2. The method of claim 1, wherein said first surfactant at said interface prevents said second surfactant from flowing from said first fluid phase to said second fluid phase.

3. The method of claim 1, wherein said first surfactant is a di-block copolymer comprising a perfluorinated polyether block bonded to a polyethylene glycol block.

4. (canceled)

5. (canceled)

6. (canceled)

7. The method of claim 3, wherein said di-block copolymer is a compound of Formula II: 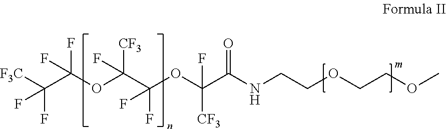
wherein m is an integer from 5 to 50 and n is an integer from 5 to 60.

8. (canceled)

9. The method of claim 1, wherein said second surfactant is n-dodecyl-D-maltoside.

10. (canceled)

11. (canceled)

12. The method of claim 1, wherein said plurality of droplets comprises particles with nucleic acid barcodes.

13. The method of claim 12, wherein said particles are beads.

14. (canceled)

15. (canceled)

16. (canceled)

17. The method of Claim 1, wherein said first fluid phase is an aqueous phase and said second fluid phase is a nonaqueous phase, and wherein said non-aqueous phase comprises a fluorinated oil.

18. (canceled)

19. The method of claim 1, wherein said plurality of droplets comprises nucleic acid molecules.

20. (canceled)

21. The method of claim 1, wherein said plurality of droplets is generated at an intersection of at least a first channel and a second channel, wherein said first fluid phase or said second fluid phase, but not both, is directed along said first channel.

22. A system for forming an emulsion comprising a plurality of droplets, comprising a droplet generator that is configured to generate an emulsion comprising said plurality of droplets; and
a controller operatively coupled to said droplet generator, wherein said controller is programed to:
(i) bring a first fluid phase in contact with a second fluid phase that is immiscible with said first fluid phase to generate said emulsion comprising said plurality of droplets, wherein said plurality of droplets comprises (1) said first fluid phase or said second fluid phase, (2) a first surfactant at an interface between said first fluid phase and said second fluid phase, and (3) a second surfactant that is different than said first surfactant; and
(ii) upon generating at least a subset of said plurality of droplets, (1) direct collection of said plurality of droplets or (2) direct said plurality of droplets along a channel, wherein upon collecting or directing said plurality of droplets along said channel, at most 5% of said plurality of droplets coalesce.

23. The system of claim 22, wherein said first surfactant at said interface prevents said second surfactant from flowing from said first fluid phase to said second fluid phase.

24. The system of claim 22, wherein said first surfactant is a di-block copolymer comprising a perfluorinated polyether block bonded to a polyethylene glycol block.

25. (canceled)

26. (canceled)

27. (canceled)

28. The system of claim 24, wherein said di-block copolymer is a compound of Formula II: 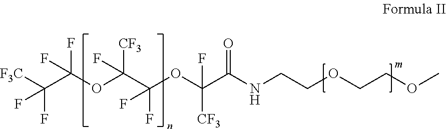
wherein m is an integer from 5 to 50 and n is an integer from 5 to 60.

29. (canceled)

30. The system of claim 22, wherein said second surfactant is n-dodecyl-D-maltoside.

31. (canceled)

32. (canceled)

33. The system of claim 22, wherein said plurality of droplets comprises particles with nucleic acid barcodes.

34. The system of claim 33, wherein said particles are beads.

35. (canceled)

36. (canceled)

37. (canceled)

38. The system of claim 22, wherein said first fluid phase is an aqueous phase and said second fluid phase is a non-aqueous phase, and wherein said non-aqueous phase comprises a fluorinated oil.

39. (canceled)

40. The system of claim 22, wherein said plurality of droplets comprises nucleic acid molecules.

41. (canceled)

42. The system of claim 22, wherein said plurality of droplets is generated at an intersection of at least a first channel and a second channel, wherein said first fluid phase or said second fluid phase, but not both, is directed along said first channel.

43. (canceled)

44. The method of claim 13, wherein said beads are gel beads.

45. The method of claim 34, wherein said beads are gel beads.
